(19) United States
(12) Patent Application Publication
(10) Pub. No.: US 2026/0196331 A1
(43) Pub. Date: Jul. 9, 2026

(54) Computer-Implemented System and Method for Personalized Ingredient Analysis

(71) Applicant: Ingredient IQ LLC, Frankfort, IL (US)

(72) Inventors: Quentien Marcell Brewington, Frankfort, IL (US); Tammy Rasheta Brewington, Frankfort, IL (US)

(73) Assignee: Ingredient IQ LLC, Frankfort, IL (US)

(21) Appl. No.: 19/306,606

(22) Filed: Aug. 21, 2025

Related U.S. Application Data
(60) Provisional application No. 63/742,074, filed on Jan. 6, 2025.

(51) Int. Cl. G16H 20/60; G06F 40/284; G06K 7/10; G06K 7/14; G06V 30/10

(52) U.S. Cl. CPC G16H 20/60; G06K 7/10366; G06K 7/1413; G06K 7/1417; G06F 40/284; G06V 30/10

(57) ABSTRACT

A computer-implemented method and system for providing personalized ingredient analysis for food products includes receiving user health profile data including dietary restrictions, allergies, or health conditions, and capturing ingredient data from a food product using an image capture device, barcode scanner, or RFID tag reader associated with a computing device. The method derives ingredient information from the captured ingredient data representing a plurality of identified ingredients through optical character recognition processing, QR code analysis, barcode scanning, or RFID tag identification. An artificial intelligence system trained on nutritional and health data analyzes the ingredient information to determine health implications. The method compares the health implications against the user health profile data to produce comparison output, generates and provides user output based on the comparison output that includes a visual Go/No-Go indicator representing compatibility between the user health profile data and the food product or identified ingredients.

### CROSS-REFERENCE TO RELATED APPLICATIONS

This application claims priority to U.S. Prov. Pat. App. No. 63/742,074, entitled, “Artificial Intelligence Health and Nutrition System,” filed on Jan. 6, 2025, which is incorporated by reference herein.

### BACKGROUND

Currently, many individuals struggle to assess whether specific ingredients align with their dietary restrictions, health conditions, or overall wellness goals due to the complexity of ingredient lists and the absence of personalized insights. These issues may be compounded by limited access to accurate, research-backed health data while shopping or making food choices and the inability to decipher information quickly and accurately from packaging information, such as ingredients.

Existing applications allow users to enter packaging information from foods manually, but they often lack personalized health integration and rely on generic ingredient assessments, failing to account for individual dietary needs, allergies, and health conditions. Additionally, these applications frequently provide limited data accuracy and do not adapt to user preferences or leverage trusted health sources for research-backed insights.

As a result, there is a need for improved techniques for making nutrition-based dietary recommendations.

### SUMMARY

Embodiments of the present invention provide a computer-implemented system and method for personalized ingredient analysis that addresses the limitations of existing nutritional assessment applications. The system may capture ingredient data from food products using multiple input modalities, including image capture devices, barcode scanners, and RFID tag readers, enabling users to quickly obtain ingredient information without manual data entry. An artificial intelligence analysis module may process the captured ingredient information using natural language processing models and machine learning algorithms trained on nutritional and health data to determine comprehensive health implications for each identified ingredient.

The system may compare these health implications against personalized user health profile data that includes dietary restrictions, allergies, and health conditions to generate tailored compatibility assessments. Unlike conventional applications that provide generic ingredient evaluations, embodiments of the present invention may offer individualized recommendations that account for specific user health requirements, medication interactions, and dietary preferences. The system may generate visual Go/No-Go indicators that provide immediate guidance regarding food product compatibility, enabling users to make informed dietary decisions quickly and confidently.

Embodiments of the system may integrate with trusted health databases and authoritative medical sources to ensure that health implications are based on current research and regulatory information. The artificial intelligence components may continuously learn and adapt based on user feedback, improving the accuracy and relevance of future recommendations. The system may also provide detailed explanations of ingredient health impacts, alternative product suggestions, and educational information to support user understanding of nutritional choices.

In some embodiments, a computer-implemented method for providing personalized ingredient analysis may receive user health profile data including dietary restrictions, allergies, and health conditions, and may capture ingredient data from a food product using an image capture device, barcode scanner, and RFID tag reader associated with a computing device. The method may derive ingredient information from the captured ingredient data representing a plurality of identified ingredients through optical character recognition processing on captured image data, QR code analysis, barcode scanning, and RFID tag identification. An artificial intelligence system comprising a natural language processing model and machine learning model trained on nutritional and health data may analyze the ingredient information to determine health implications. The method may compare the health implications against the user health profile data to produce comparison output, may generate an output based on the comparison output that includes a visual Go/No-Go indicator representing compatibility between the user health profile data and the food product and identified ingredients, and may provide the output to a user.

Other features and advantages of various aspects and embodiments of the present invention will become apparent from the following description and from the claims.

### BRIEF DESCRIPTION OF THE DRAWINGS

FIG. 1 is a flow diagram of a method of nutrition analysis according to aspects of the present invention;

FIG. 2 is a swim lane diagram of a system of nutrition analysis according to aspects of the present invention;

FIG. 3 is an additional swim lane diagram of a system of nutrition analysis according to aspects of the present invention;

FIG. 4 is a flow diagram of a method for providing personalized ingredient analysis according to aspects of the present invention; and

FIG. 5 is a block diagram of a personalized ingredient analysis system according to aspects of the present invention.

### DETAILED DESCRIPTION

Current systems for providing nutritional assistance to users provide one-size-fits-all assessments, ignoring individual health profiles and dietary needs. These systems often lack reliable integration with credible health data sources, leading to inaccurate or incomplete ingredient analyses that fail to support informed decision-making.

Broadly, an embodiment of the present invention improves existing systems by offering AI-driven, personalized ingredient analysis tailored to individual health profiles, including allergies and dietary restrictions. Embodiments of the system of the present invention integrates trusted health data sources, such as the Mayo Clinic and the National Institutes of Health (NIH), providing accurate, research-backed insights and adaptive learning for continuously refined recommendations. Embodiments of the system of the present invention combine real-time ingredient analysis with AI-driven personalized health recommendations, adaptive learning, and integration with trusted health databases like Mayo Clinic and NIH. Unlike existing systems, embodiments of the present invention offer tailored insights aligned with user profiles, providing an intuitive Go/No-Go indicator for effortless decision-making.

Broadly, embodiments of the present invention provide a computer-implemented method for consolidating and visually presenting information related to the overall health of the user.

Broadly, embodiments of the present invention may include one or more servers and at least one computer. Each server and computer in embodiments of the present invention may include computing systems. This disclosure contemplates any suitable number of computing systems. This disclosure contemplates the computing system taking any suitable physical form. For example, and not by way of limitation, the computing system may be a virtual machine (VM), an embedded computing system, a system-on-chip (SOC), a single-board computing system (SBC) (e.g., a computer-on-module (COM), or system-on-module (SOM)), a desktop computing system, a laptop or notebook computing system, a smartphone, an interactive kiosk, a mainframe, a mesh of computing systems, a server, an application server, or a combination of two or more of these. Where appropriate, the computing systems may include one or more computing systems, be unitary or distributed, span multiple locations, span multiple machines, or reside in a cloud, which may include one or more cloud components in one or more networks. Where appropriate, one or more computing systems may perform without substantial spatial or temporal limitation of one or more steps of one or more methods described or illustrated herein. As an example, and not by way of limitation, one or more computing systems may perform in real-time or batch mode one or more steps of one or more methods described or illustrated herein. One or more computing systems may perform at different times or different locations, one or more steps of one or more methods described or illustrated herein, where appropriate.

In certain embodiments, the network may refer to any interconnecting system capable of transmitting audio, video, signals, data, messages, or any combination of the preceding. The network may include all or a portion of a public switched telephone network (PSTN), a public or private data network, a local area network (LAN), a metropolitan area network (MAN), a wide area network (WAN), a local, regional, or global communication or computer network such as the Internet, a wireline or wireless network, an enterprise intranet, or any other suitable communication link, including combinations thereof.

In some embodiments, the computing systems may execute any suitable operating system such as IBM's zSeries/Operating System (z/OS), MS-DOS, PC-DOS, MAC-OS, WINDOWS, UNIX, OpenVMS, an operating system based on LINUX, or any other appropriate operating system, including future operating systems. In some embodiments, the computing systems may be a web server running web server applications such as Apache, Microsoft's Internet Information Server™, and the like.

In particular embodiments, the computing systems include a processor, a memory, a user interface, and a communication interface. In particular embodiments, the processor includes hardware for executing instructions, such as those making up a computer program. The memory includes main memory for storing instructions such as computer program(s) for the processor to execute or data for a processor to operate on. The memory may include mass storage for data and instructions such as the computer program. As an example, and not by way of limitation, the memory may include an HDD, a floppy disk drive, flash memory, an optical disc, a magneto-optical disc, magnetic tape, a Universal Serial Bus (USB) drive, a solid-state drive (SSD), or a combination of two or more of these. The memory may include removable or non-removable (or fixed) media, where appropriate. The memory may be internal or external to a computing system, where appropriate. In particular embodiments, the memory is a non-volatile, solid-state memory.

The user interface includes hardware, software, or both, providing one or more interfaces for communication between a person and the computer systems. As an example, and not by way of limitation, a user interface device may include a keyboard, keypad, microphone, monitor, mouse, printer, scanner, speaker, still camera, stylus, tablet, touchscreen, trackball, video camera, another suitable user interface or a combination of two or more of these. A user interface may include one or more sensors. This disclosure contemplates any suitable user interface and any suitable user interfaces for them.

The communication interface includes hardware, software, or both, providing one or more interfaces for communication (e.g., packet-based communication) between the computing systems over the network. As an example, and not by way of limitation, the communication interface may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and communication interface. As an example, and not by way of limitation, the computing systems may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, the computing systems may communicate with a wireless PAN (WPAN) (e.g., a BLUETOOTH WPAN), a WI-FI network, a WI-MAX network, a cellular telephone network (e.g., a Global System for Mobile Communications (GSM) network), or other suitable wireless network or a combination of two or more of these. The computing systems may include any suitable communication interface for any of these networks, where appropriate.

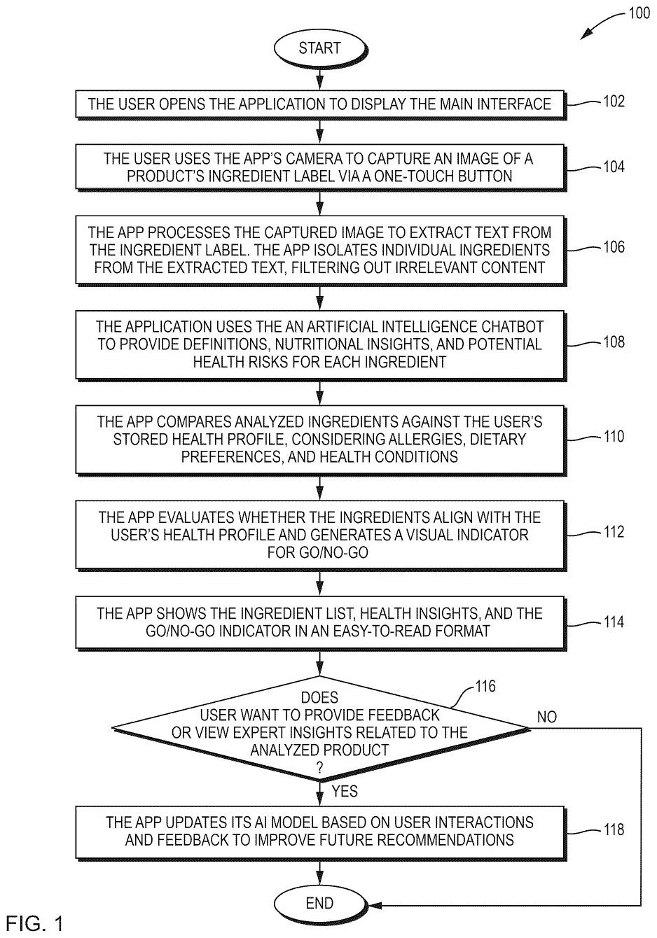

Referring now to FIG. 1, a flow diagram of a method 100 of nutritional assistance is illustrated. In embodiments, a user may accomplish a plurality of steps before initiating the method of FIG. 1. In embodiments, the user can launch a nutritional assistance application on a computing device, such as a smartphone. Once launched, the user can log in or create a user account if they are using it for the first time. Once the user profile is created or logged into, the user can set up a health profile by navigating to the profile section of the application and inputting personal health details, including, but not limited to, dietary restrictions (e.g., vegetarian, gluten-free), allergies (e.g., peanuts, lactose), health conditions (e.g., diabetes, hypertension), etc. Once the health profile is created, the user can save preferences to enable tailored ingredient analysis. The method can begin when a user navigates on their computing device to display a main interface of a nutritional assistance application.

Once the profile and health information are created and saved, the method 100 of FIG. 1 may begin with the user accessing the main display interface of the nutritional assistance application (FIG. 1, operation 102). Once on the main display interface, the user may capture an ingredient label utilizing an input device on the computing device (FIG. 1, operation 104). In embodiments, the ingredient label can be captured via a camera of the computing device by actuating a user interface element on the main display interface. In embodiments, the user can position the camera of the computing device to focus on the ingredient list and capture an image.

Once captured, the nutritional assistance application performs image processing to extract text from the ingredient, isolating individual ingredients from the extracted text, and filtering out irrelevant content (FIG. 1, operation 106). In embodiments, image processing to extract text can include optical character recognition (OCR) and/or other image processing algorithms for text extraction known in the art.

Once the text is extracted, individual ingredients are parsed and analyzed by an AI module, which interprets each ingredient and identifies potential health implications. In embodiments, algorithms such as an artificial intelligence chatbot provide definitions, nutritional insights, and potential health risks for each ingredient (FIG. 1, operation 108). Concurrently, or after AI analysis, each ingredient can be compared to the user's profile, such as the health profile, to match known allergies, dietary preferences, and health conditions (FIG. 1, operation 110).

After each ingredient is compared to the user's health profile, a determination is made as to whether each ingredient aligns with the user's health profile or not, and a go/no-go indicator is generated (FIG. 1, operation 112). In embodiments, a go indicator is indicative of alignment with the user's health profile, while a no-go indicator is indicative of a lack of alignment with the user's health profile.

Once all analysis is performed, a unified display is provided to the user (FIG. 1, operation 114). In embodiments, the unified display can include ingredients list(s), health insight(s), and/or a go/no-go indicator. In embodiments, the unified display can include Ingredient definitions, nutritional insights, linked references (e.g., Mayo Clinic, NIH), and flagged items for risks or restrictions.

Optionally, a user can provide feedback on the nutritional application (FIG. 1, operation 116), which can be used by the AI-module of the application to improve future insights, recommendations, and/or suggestions (FIG. 1, operation 118). In embodiments, feedback can include one or more user interactions with the application.

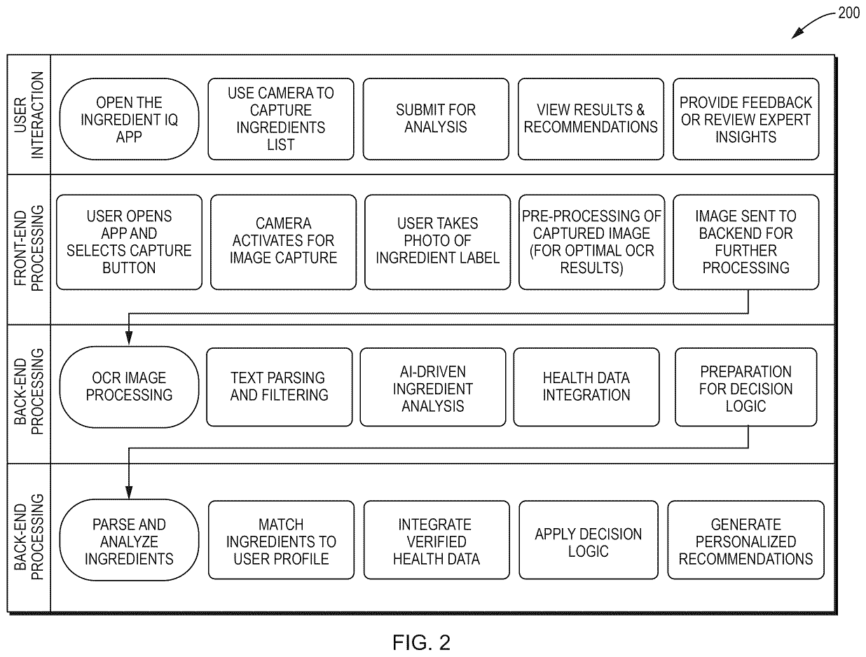

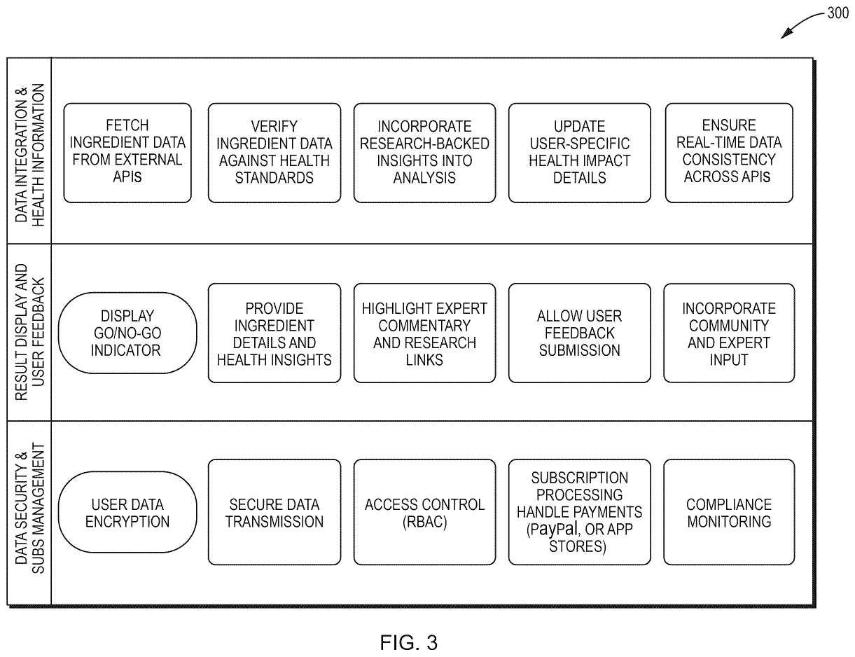

Referring now to FIGS. 2-3, aspects of a nutritional assistance system 200 and 300, respectively, are illustrated. The systems 200 and 300 of FIGS. 2 and 3 may be implemented within a single system or divided among multiple systems in any of a variety of ways. In embodiments, the nutritional assistance system of the present invention may include a plurality of components, each having one or more functionalities. In embodiments, the plurality of components can include, but are not limited to: a user Interface (UI) configured to allow users to interact with an application of the nutritional assistance system, capture images, input health profiles, and view results; a Camera device and module configured to capture clear images of ingredient labels for analysis; an OCR Processing System configured to extract text from images accurately; an AI Analysis Engine configured to analyze ingredients and provide health insights and additional information based on the analysis; a Health Data Integration Module configured to gather, evaluate, and ensure the accuracy and credibility of ingredient information through trusted sources; a User Profile Matching System configured to tailor results to individual health needs; a Decision Logic Framework configured to generate Go/No-Go recommendations based on user profiles; a Data Security System configured to ensure user data privacy and compliance with regulations.

Additionally, a number of optional components can be provided in the system of the present invention, including, but not limited to: a Feedback and Expert Insights Module configured to improve personalization and community engagement; an Adaptive Learning System configured to enhance user experience; a Subscription and Payment Gateway configured for accessing premium features or monetization; an Ingredient Comparison Feature configured to allow users to compare multiple products side by side for more informed decisions; a Barcode Scanning module configured to provide barcode recognition for faster product identification; a Recipe Suggestions module configured to provide meal ideas based on user health profiles and safe ingredients; a Language Support module configured to enable multilingual support for international users; and an Offline Mode configured to allow users to store previous analyses or perform limited functionality without internet access.

In embodiments, components can work together to provide nutritional assistance. For example, the user may command the camera module and device through the User Interface to capture the ingredient label image. The captured image may be processed by the OCR Processing System, which extracts text for the Ingredient Parsing Engine. Parsed data may be analyzed by the AI Analysis Engine, integrating insights from the Health Data Integration Module. The User Profile Matching System may check the parsed data against personalized profiles, using feedback from the Decision Logic Framework to generate Go/No-Go results. Results may then be displayed on the UI, with users optionally contributing via the Feedback and Expert Insights Module.

In embodiments, all data may be secured by the Data Security and Privacy System, and subscription payments may be managed through the Subscription and Payment Gateway. The Backend Infrastructure may ensure the smooth operation of all systems, linking frontend and backend functions seamlessly.

In terms of data programmatic flow, embodiments of the system of the present invention may include input flow, output flow, and data flow. In embodiments, input flow may include user interactions, which may begin at the UI and lead to data capture via the camera module and subsequent processing by the OCR processing system and ingredient parsing engine. In embodiments, output flow includes the decision logic framework, which may generate results that are displayed on the UI. Additionally, Feedback and expert insights may update the adaptive learning cycle through the Feedback and Expert Insights Module. Finally, Data Flow may include all components secured by the Data Security and Privacy System, while the Subscription and Payment Gateway manages access to premium features. In embodiments, the system infrastructure can include the Backend Infrastructure, which connects all modules, ensuring real-time processing and scalability.

The components of the system of the present invention function collaboratively to deliver real-time, personalized ingredient analysis, enabling informed food choices for users. The UI may provide a user-friendly platform for capturing images, managing profiles, and viewing results. In embodiments, the UI initiates interaction with the system by enabling users to interact with the application and access its features. Additionally, the UI interfaces with the Camera Module to capture high-quality images and send them for processing.

The Camera Module may utilize the camera, or image capture device, of the computing device to capture clear images of ingredient labels. In embodiments, the camera module may provide processing of captured images to ensure the captured images are suitable for accurate text extraction. Additionally, the camera module, after processing, may pass the captured images to the OCR Processing System for analysis.

The OCR Processing System may extract text from the captured images using an application programming interface or other functionality, such as Google Vision API, thereby handling various label formats and fonts. In embodiments, OCR converts any text in the capture images to text for further processing. Once OCR is completed, the OCR processing system may send the text to the Ingredient Parsing Engine (4) for filtration and structuring.

The Ingredient Parsing Engine may process extracted text to identify and isolate individual ingredients from the text, removing non-relevant content like brand names. Once identified, ingredient data may be output to the AI Analysis Engine for detailed examination. An AI Analysis Engine may process ingredient data using Large Language Models or other artificial intelligence, such as generative AI, including but not limited to ChatGPT. The AI Analysis engine may utilize the ingredient data to provide additional data such as definitions, nutritional insights, and potential health implications. The Health Data Integration module may cross-reference results from the AI Analysis engine for credible insights. The health data integration module may utilize APIs to access data from scientific and medical providers, such as the Mayo Clinic, NIH, and WebMD, to validate and enhance ingredient information with reliable, research-backed data. All of the above information or data may be provided to the User profile matching System.

The User Profile Matching System may compare analyzed ingredients against user health profiles, considering allergies, dietary restrictions, and health conditions, and send results to the Decision Logic Framework for evaluation. In an exemplary embodiment, matching may be performed as follows: IF an ingredient matches an allergy or restriction, THEN flag the ingredient. All flagged ingredients can then be sent to the Decision Logic Framework.

The Decision Logic Framework may apply rules-based logic to determine whether the ingredients align with the user's profile, generating a Go/No-Go indicator. In an exemplary embodiment, the Go/No-Go Logic may be implemented as follows: IF ingredients align with the user profile, THEN display “Go”; otherwise, display “No-Go.” Results can be displayed on the UI.

The Feedback and Expert Insights Module optionally allows users to provide feedback and view expert commentary. For example, IF the user flags specific ingredients often, THEN refine sensitivity checks. Update analysis based on user feedback trends. This data improves the system's adaptive learning capabilities and works with the AI Analysis Engine to refine future recommendations.

The Data Security and Privacy System may encrypt and store user profiles and interaction data securely, ensuring compliance with GDPR, CCPA, and healthcare privacy regulations such as HIPAA. In some embodiments, the system may utilize HIPAA-compliant cloud infrastructure, such as AWS HIPAA-eligible services, and may establish business associate agreements (BAAs) with cloud service providers to ensure appropriate safeguards for protected health information. In embodiments, the data security and privacy system may protect all data interactions across components. The Subscription and Payment Gateway may manage secure subscription payments, ensuring seamless access to premium features. The gateway may integrate with the Backend Infrastructure for processing. The Backend Infrastructure acts as the backbone, connecting all components, managing data flows, and ensuring scalability and reliability. Additionally, Backend Infrastructure supports the operation of all modules, linking frontend and backend systems seamlessly.

In embodiments, elements and components may be reconfigured. For example, barcode scanning may be integrated into the system. Instead of relying solely on the OCR Processing System, a barcode scanner may replace or complement it. This reconfiguration would allow users to scan product barcodes to retrieve pre-stored ingredient information from a database, bypassing the need for text extraction.

Standalone AI Processing may be provided so that the AI Analysis Engine may be decoupled from real-time OCR and used with pre-existing ingredient databases. This would remove reliance on the Camera Module but still provide health insights based on stored product data.

Direct Integration for Ingredient Data may be provided such that the Health Data Integration Module may be configured to retrieve entire product ingredient lists directly from manufacturers or external databases, skipping the Image Capture and OCR Processing steps altogether.

Local Device Processing may be provided such that instead of using cloud-based infrastructure for AI and OCR processing, these functions could be executed locally on the user's device. This reconfiguration reduces reliance on the Backend Infrastructure while still providing similar results.

The User Profile Matching System may be swapped with a simple keyword flagging system that identifies allergens or restricted ingredients without requiring a detailed health profile. This may simplify user profile matching, thereby reducing computational loads and speeding system processing.

Static ingredient analysis may be provided so that the decision logic framework may work with pre-defined health rules instead of adaptive learning. This approach may provide similar Go/No-Go recommendations but without dynamic updates based on user feedback.

The systems and methods of embodiments of the present invention may be used in various ways across different fields of technology to benefit larger systems. For example, embodiments of the present invention may be integrated into electronic health record (EHR) systems, where the system may analyze patient dietary needs alongside prescribed medications to recommend safe food options in hospitals or clinics. The system may be embedded into smart shopping carts or self-checkout systems, where it may provide real-time ingredient analysis and personalized product recommendations while customers shop. The system may be used in manufacturing plants, where it may verify compliance with food safety standards, ensuring that ingredients meet regulatory and consumer-specific standards. The system may be integrated into smart refrigerators or kitchen assistants, where it could analyze stored food items, flagging potential allergens or unsuitable ingredients based on household preferences. The system may be adapted for schools, and the system may educate children on nutrition by analyzing cafeteria menus and explaining the health impacts of ingredients. The system may be integrated into sustainability-focused industries, where it may assess products for eco-friendly and ethical ingredients, helping businesses align with green initiatives. The system may be used in humanitarian efforts, where it may assist in distributing food that meets the nutritional and dietary needs of specific populations in disaster-stricken or underserved areas. By applying the systems core technology in these systems, it extends its utility beyond personal use, offering innovative solutions for healthcare, retail, education, sustainability, and humanitarian efforts.

The system of the present invention may produce a variety of products. For example, Personalized Health Reports may be generated for individual users based on dietary preferences, allergies, and health conditions. These reports may include detailed ingredient analyses, flagged risks, and nutritional insights.

An Ingredient Knowledge Database may be provided, which includes a centralized, curated database of ingredients with associated definitions, health impacts, and research-backed insights. Custom Meal Plans and Shopping Lists may be provided by analyzing user profiles and flagged ingredients. Tailored recommendations for meals or products to meet individual health goals can also be provided.

Smart Retail Tools may be provided and integrated into kiosks or shopping apps to analyze ingredient data in-store and recommend healthier alternatives. Educational Materials such as infographics, e-books, or guides that educate users about ingredient safety, dietary compatibility, and nutrition may be provided.

User Feedback Insights may be provided, which are aggregated feedback from users that contributes to a community-driven resource for a better understanding of ingredient impacts. Product Safety Certifications may be generated for food or cosmetic manufacturers to verify and market products as allergy-safe, compliant with dietary needs, or environmentally friendly. Global Ingredient Trends and Analytics may be provided, and they can provide data-driven insights about ingredient use and consumer preferences, which is beneficial for manufacturers and researchers. Research datasets may be provided as anonymized data on ingredient analyses and user interactions, which are useful for scientific studies or market analysis. Adaptive Learning Models may be provided and may continually improve AI models tailored to individual and collective user needs, which may be applied in other health or safety-related domains.

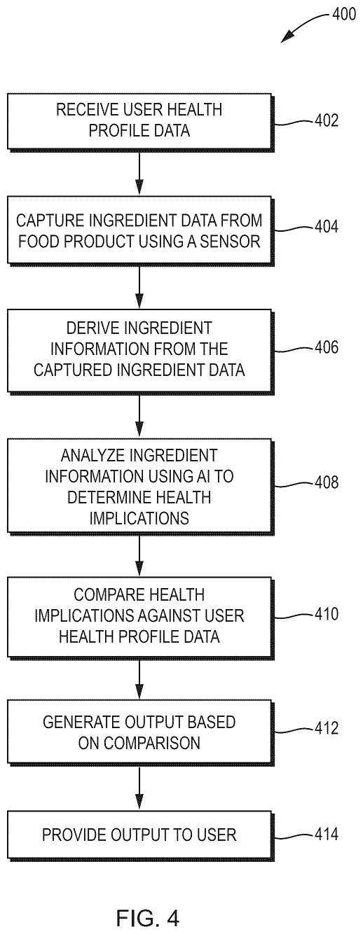

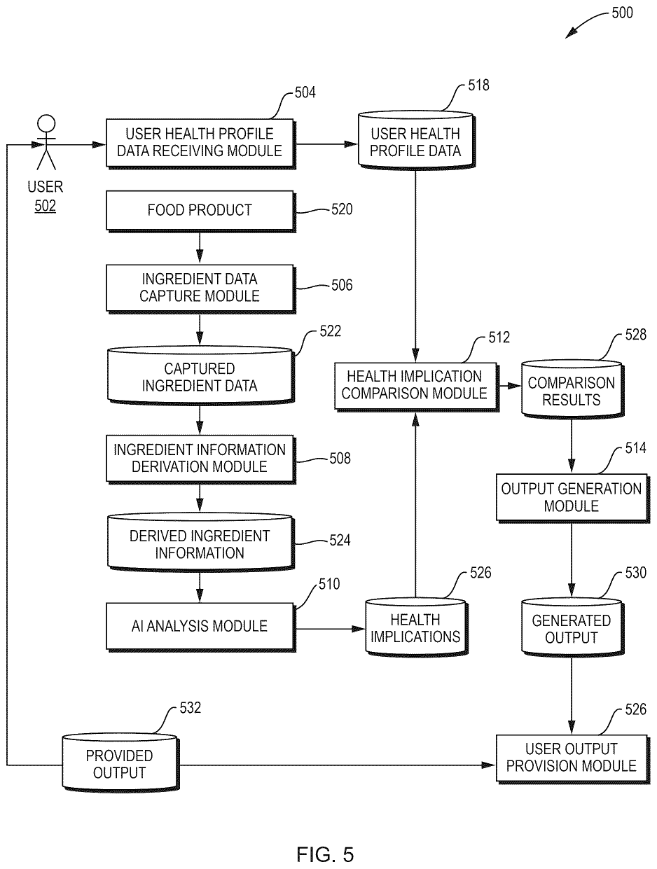

Referring to FIG. 4, a flowchart is shown of a method 400 for providing personalized ingredient analysis according to one embodiment of the present invention. Referring to FIG. 5, a block diagram is shown of a personalized ingredient analysis system 500 according to one embodiment of the present invention.

Referring to FIG. 4, the method 400 begins with operation 402, which involves receiving user health profile data 518 associated with the user 502. The user health profile data 518 may, for example, include dietary restrictions, allergies, and/or health conditions. As shown in FIG. 5, this operation may be performed by the user health profile data receiving module 504 of the personalized ingredient analysis system 500. The user 502 may interact with the user health profile data receiving module 504 to input and store personal health information, in the user health profile data 518, that forms the foundation for subsequent personalized ingredient analysis.

Referring to FIG. 5, the user health profile data 518 may include any of a variety of information about the user's dietary restrictions, allergies, and health conditions. In some embodiments, the user health profile data 518 may include dietary restrictions such as vegetarian, vegan, gluten-free, ketogenic, paleo, Mediterranean, low-FODMAP, dairy-free, and/or other specific dietary preferences. These dietary restrictions may encompass, for example, voluntary lifestyle choices and/or medically-prescribed dietary limitations that affect food selection. For example, the user health profile data 518 may include information about a low-sodium diet requirement due to hypertension, carbohydrate restrictions for diabetes management, and/or oxalate limitations for kidney stone prevention. The user health profile data 518 may include religious or cultural dietary practices such as kosher, halal, Hindu vegetarianism, and/or Buddhist dietary restrictions that influence ingredient acceptability.

The user health profile data 518 may include allergy information, such as food allergies and/or sensitivities. In some cases, the allergy data may specify common allergens such as peanuts, tree nuts, shellfish, dairy, eggs, soy, wheat, and/or fish. The user health profile data 518 may include severity levels for different allergies, ranging from mild sensitivities that cause discomfort to severe allergies that may trigger anaphylactic reactions. These severity levels may differ between different users 502, as individual users may have varying degrees of sensitivity to the same allergens. The user health profile data 518 may include less common allergies or intolerances, such as histamine intolerance, sulfite sensitivity, food colorings, preservatives, and/or specific additive sensitivities like monosodium glutamate (MSG) or aspartame. The user health profile data 518 may categorize allergic reactions by symptom type, such as gastrointestinal distress, skin reactions, respiratory issues, and/or systemic responses, providing a comprehensive allergenic profile for each user 502. In some embodiments, the user health profile data 518 may include threshold information indicating minimum quantities of allergens that trigger reactions for the user 502.

The user health profile data 518 may include information about health conditions that influence dietary choices and ingredient compatibility. These health conditions may include metabolic disorders such as diabetes, insulin resistance, and/or hypoglycemia; cardiovascular conditions such as hypertension, hyperlipidemia, and/or congestive heart failure; digestive system disorders such as irritable bowel syndrome, Crohn's disease, ulcerative colitis, celiac disease, and/or gastroesophageal reflux disease; renal conditions such as chronic kidney disease and/or kidney stones; hepatic conditions such as fatty liver disease and/or hepatitis; neurological conditions such as migraine, epilepsy, and/or multiple sclerosis; endocrine disorders such as hypothyroidism, hyperthyroidism, and/or adrenal insufficiency; and autoimmune conditions such as rheumatoid arthritis, lupus, and/or psoriasis. In some embodiments, the user health profile data 518 may include information about medications the user 502 is taking, as certain ingredients may interact with pharmaceutical treatments. For example, the user health profile data 518 may indicate that a user is taking blood thinners and therefore needs to monitor vitamin K intake, that a user is on certain antidepressants and needs to avoid tyramine-rich ingredients, and/or that a user is taking antibiotics that interact with calcium-rich foods.

The user health profile data 518 may include nutritional goals and requirements based on the user's physiological status. These may include macronutrient targets such as protein requirements for athletes or pregnant women, carbohydrate limitations for weight management, and/or fat composition preferences for heart health. Micronutrient considerations in the user health profile data 518 may include iron needs for those with anemia, calcium requirements for those with osteoporosis risk, sodium restrictions for hypertension management, and/or potassium monitoring for those with kidney dysfunction. In some embodiments, the user health profile data 518 may include life stage information such as pregnancy, lactation, childhood, adolescence, and/or advanced age, each with specific nutritional implications. The user health profile data 518 may include physical activity levels, basal metabolic rate, and/or weight management goals that influence caloric and nutrient requirements.

Referring again to FIG. 5, the user health profile data 518 may include personal ingredient preferences and aversions that are not medically necessary but influence food choices. These preferences may include taste aversions to specific flavors or ingredients, texture preferences, and/or ingredient avoidances based on personal values such as sustainability concerns, animal welfare considerations, and/or genetic modification avoidance. The user health profile data 518 may include information about previous adverse reactions to specific ingredients that do not qualify as allergies but warrant caution. In some embodiments, the user health profile data 518 may include family health history information that may indicate genetic predispositions requiring preventative dietary measures, such as familial hypercholesterolemia suggesting reduced saturated fat intake and/or family history of diabetes suggesting careful carbohydrate monitoring.

The user health profile data 518 may include temporal dietary considerations that affect ingredient compatibility at specific times. These temporal factors may include time-restricted eating windows, seasonal allergies that affect food sensitivities during certain months, cyclical dietary patterns related to hormonal fluctuations, and/or temporary dietary protocols such as elimination diets, detoxification programs, and/or pre-surgical nutrition plans. In some cases, the user health profile data 518 may include medication schedules that influence optimal timing for certain nutrients or ingredients. For example, the user health profile data 518 may indicate that iron supplements should not be taken with calcium-rich foods, and/or that certain medications should be taken with or without food containing specific ingredients.

As described in more detail below, the method 400 and system 500 may compare the user health profile data 518 to the health implications 526 to generate comparison results 528. The user health profile data 518 may have any of a variety of characteristics which facilitate that comparison. For example, the user health profile data 518 may include structured data formats and/or standardized coding systems that facilitate efficient comparison operations in operation 410. In some embodiments, the user health profile data 518 may utilize standardized medical coding systems such as ICD-10 codes for health conditions, SNOMED CT codes for clinical terminology, and/or RxNorm codes for medications, enabling precise matching algorithms during the comparison process. The user health profile data 518 may include hierarchical categorization structures that organize related conditions, allergies, and dietary restrictions into parent-child relationships, allowing the health implication comparison module 512 to evaluate both specific ingredients and broader ingredient categories simultaneously.

The user health profile data 518 may include quantitative thresholds and/or tolerance levels for various ingredients, enabling the comparison process to evaluate not only the presence or absence of problematic ingredients but also their concentration levels within food products. In some cases, the user health profile data 518 may specify minimum trigger amounts for allergens, maximum daily intake limits for restricted substances, and/or optimal ranges for beneficial nutrients based on individual health requirements. These quantitative thresholds and tolerance levels may vary between different users 502, as individual health conditions, genetic factors, and/or physiological responses create unique sensitivity profiles that require or otherwise benefit from personalized threshold parameters. The user health profile data 518 may include interaction matrices that pre-define relationships between different health conditions, medications, and ingredient categories, streamlining the comparison process by providing pre-computed compatibility assessments.

The user health profile data 518 may include priority weighting systems that assign relative importance scores to different health considerations, allowing the health implication comparison module 512 to appropriately balance competing health factors when generating recommendations. For example, the user health profile data 518 may assign higher priority weights to life-threatening allergies compared to dietary preferences, ensuring that critical health risks receive appropriate emphasis during the comparison process. These priority weighting systems may differ between different users 502, as individual users may have varying combinations of health conditions that require unique prioritization schemes to ensure appropriate risk assessment and recommendation generation. The user health profile data 518 may include temporal validity periods for different health information elements, enabling the comparison process to consider the current relevance of various health conditions and dietary requirements.

The user health profile data 518 may include cross-reference mappings that link ingredient names to their chemical compositions, alternative names, and/or derivative compounds, enhancing the health implication comparison module 512's ability to identify potentially problematic ingredients that may be listed under different nomenclatures. In some embodiments, the user health profile data 518 may include ingredient sensitivity profiles that specify individual reactions to ingredient combinations or processing methods, enabling more sophisticated analysis of complex food products during the comparison step. The user health profile data 518 may include machine-readable flags/tags that enable rapid filtering and sorting operations, reducing computational overhead during the comparison process while maintaining comprehensive health assessment capabilities.

The user health profile data receiving module 504 may implement various techniques for receiving and/or generating the user health profile data 518 based on input from the user 502 and/or one or more other sources. In some embodiments, the user health profile data receiving module 504 may present guided questionnaires that walk users through different categories of health information, ensuring thorough data collection while maintaining user-friendly interaction. The user health profile data receiving module 504 may provide options for users to upload medical records or import health data from other applications or devices, streamlining the profile creation process. For example, the user health profile data receiving module 504 may integrate with electronic health record (EHR) systems, healthcare provider portals, fitness trackers, smartwatches, nutrition apps, and/or genetic testing services to automatically import relevant health information with user authorization. When integrating with healthcare systems, the module 504 may utilize HIPAA-compliant data exchange protocols and may establish business associate agreements with healthcare providers to ensure appropriate handling of protected health information. The user health profile data receiving module 504 may connect to pharmacy databases to retrieve medication history and prescription information, which may be analyzed to identify potential ingredient interactions.

The user health profile data 518 may not be limited to containing manually-input data from the user 502. The user health profile data receiving module 504 may process input from the user 502 in various ways before storing the resulting processed data in the user health profile data 518. For instance, the user health profile data receiving module 504 may employ natural language processing algorithms to extract relevant health information from free-text descriptions provided by the user 502. The user health profile data receiving module 504 may utilize machine learning models to categorize and standardize user-reported symptoms or conditions according to established medical taxonomies such as ICD-10 or SNOMED CT. In some cases, the user health profile data receiving module 504 may implement inference engines that identify potential allergies or sensitivities based on reported symptom patterns, even when the user 502 has not explicitly identified these connections.

The user health profile data receiving module 504 may leverage generative AI technologies to enhance and expand the user health profile data 518. For example, large language models may be employed to translate technical medical terminology into standardized database entries, ensuring consistency across different input sources. Generative AI may analyze patterns in the user's reported health history to suggest additional health factors that might be relevant but were not explicitly mentioned by the user 502. In some embodiments, the user health profile data receiving module 504 may utilize generative AI to create comprehensive health risk profiles based on limited initial inputs, which may then be verified or refined through follow-up questions with the user 502. These AI-generated insights may be clearly labeled within the user health profile data 518 to distinguish them from directly reported information.

With continued reference to FIG. 5, the user health profile data receiving module 504 may receive data from one or more sources other than the user 502 and store such data, either directly or after additional processing, in the user health profile data 518. These sources may include healthcare providers who may upload clinical notes, diagnostic test results, or treatment plans through secure healthcare information exchange protocols that comply with HIPAA requirements and utilize encrypted transmission channels with appropriate business associate agreements. Public health databases may provide regional allergen information, seasonal health advisories, or epidemiological data relevant to the user's location. Nutritional research databases may contribute evidence-based dietary guidelines specific to various health conditions. In some cases, the user health profile data receiving module 504 may access anonymized population health statistics to contextualize individual user data within broader demographic patterns, potentially identifying health factors that may be statistically relevant based on the user's age, gender, ethnicity, or geographic location.

The user health profile data receiving module 504 may implement various data processing techniques to transform raw inputs into structured, actionable health profile information. These techniques may include data normalization processes that convert diverse input formats into standardized data structures, ontology mapping that aligns user-reported terms with established medical vocabularies, and confidence scoring algorithms that assess the reliability of different data sources. The user health profile data receiving module 504 may employ temporal analysis to track changes in health status over time, identifying emerging patterns or trends in the user's health profile. In some embodiments, the user health profile data receiving module 504 may utilize differential privacy techniques when incorporating population-level data, ensuring that statistical insights are gained without compromising individual privacy. The user health profile data receiving module 504 may offer periodic prompts for users to update their health information, ensuring that the user health profile data 518 remains current and accurate as health conditions or dietary needs change over time.

Referring to FIG. 4, the method 400 continues with operation 404, which involves capturing ingredient data from a food product using at least one of an image capture device associated with a computing device, a barcode scanner associated with the computing device, an RFID tag reader associated with the computing device, a voice recognition system associated with the computing device, a manual text input interface associated with the computing device, a near field communication (NFC) reader associated with the computing device, and/or a Bluetooth Low Energy (BLE) beacon reader associated with the computing device. As shown in FIG. 5, this operation may be performed by the ingredient data capture module 506 of the personalized ingredient analysis system 500. The ingredient data capture module 506 may interface with the food product 520 to obtain ingredient information through various capture mechanisms, generating captured ingredient data 522 for subsequent processing.

In some embodiments, the ingredient data capture module 506 may utilize an image capture device, such as a camera integrated into a smartphone, tablet, and/or other computing device, to capture visual information from the food product 520. The image capture device may photograph ingredient labels, nutritional information panels, and/or other text-based information displayed on packaging of the food product 520. The ingredient data capture module 506 may provide user interface controls that allow the user 502 to position the image capture device appropriately and trigger image capture at optimal moments. For example, the module 506 may include automatic focus adjustment, lighting optimization, and/or image stabilization features to ensure clear capture of ingredient text from various packaging formats and lighting conditions.

The ingredient data capture module 506 may alternatively or additionally employ a barcode scanner associated with the computing device to capture ingredient data from the food product 520. In some cases, the barcode scanner may read standard Universal Product Code (UPC) barcodes, European Article Number (EAN) barcodes, and/or other machine-readable codes present on the food product 520 packaging. The ingredient data capture module 506 may use the scanned barcode information to retrieve pre-stored ingredient data from local databases or external product information services. This approach may provide rapid ingredient data acquisition without requiring optical character recognition processing of ingredient labels.

With continued reference to FIG. 5, the ingredient data capture module 506 may incorporate an RFID tag reader associated with the computing device to capture ingredient data from the food product 520. The RFID tag reader may detect and read radio frequency identification tags embedded in or attached to the food product 520 packaging. In some embodiments, these RFID tags may contain encoded ingredient information, nutritional data, and/or product identifiers that the ingredient data capture module 506 may decode to obtain comprehensive ingredient details. The RFID tag reader functionality may enable contactless data capture, allowing users to obtain ingredient information without direct visual access to ingredient labels or barcodes.

The ingredient data capture module 506 may alternatively or additionally employ a voice recognition system associated with the computing device to capture ingredient data from the food product 520. In some cases, the voice recognition system may utilize speech-to-text processing to convert spoken ingredient lists into structured data, which may be particularly useful for users with visual impairments or in situations where visual capture methods are impractical. The ingredient data capture module 506 may incorporate natural language processing algorithms to interpret colloquial ingredient names and convert them to standardized terminology for subsequent analysis.

The ingredient data capture module 506 may provide manual text input capabilities through a manual text input interface associated with the computing device. The manual text input interface may include keyboard interfaces, touchscreen typing, and/or stylus input methods that allow users to directly enter ingredient information when other capture methods are unavailable or when ingredient information is obtained from sources other than physical packaging, such as restaurant menus or recipe cards. The ingredient data capture module 506 may provide structured input forms that guide users through systematic entry of ingredient data, ensuring consistency and completeness of manually entered information.

The ingredient data capture module 506 may incorporate a near field communication (NFC) reader associated with the computing device to capture ingredient data from the food product 520. The NFC reader may detect and communicate with NFC-enabled food packaging or smart labels through proximity-based communication without requiring visual scanning or image processing. In some embodiments, NFC tags may contain detailed ingredient information, allergen warnings, nutritional data, and/or links to comprehensive ingredient databases that the ingredient data capture module 506 may access to obtain complete ingredient details for the food product 520.

The ingredient data capture module 506 may employ a Bluetooth Low Energy (BLE) beacon reader associated with the computing device to capture ingredient data from the food product 520. The BLE beacon reader may detect and communicate with BLE beacons associated with food products or retail environments that broadcast ingredient information, product details, and/or links to comprehensive nutritional databases. The ingredient data capture module 506 may process beacon signals to access ingredient data, enabling capture of information even when physical packaging labels are not readily accessible or when products are stored in locations where direct scanning is impractical.

The ingredient data capture module 506 may implement multiple capture modalities simultaneously or sequentially, providing flexibility in how ingredient data is obtained from different types of food products 520. In some cases, the module 506 may automatically detect which capture method is most appropriate based on the available information sources on the food product 520 packaging and the user's preferences or accessibility needs. For example, the ingredient data capture module 506 may first attempt barcode scanning for rapid data retrieval, then fall back to image capture if no readable barcode is detected, or utilize voice recognition when hands-free operation is required. The captured ingredient data 522 generated by the ingredient data capture module 506 may include raw image data, decoded barcode information, RFID tag data, voice recognition transcripts, manually entered text, NFC tag information, BLE beacon data, and/or combinations thereof, depending on the capture method employed.

The food product 520 from which ingredient data is captured may comprise various forms of information sources. In some embodiments, the food product 520 may include packaged food items such as canned goods, boxed cereals, bottled beverages, frozen meals, snack packages, and/or other commercially prepared foods that display ingredient information directly on their packaging materials. The ingredient data capture module 506 may capture ingredient information from labels, stickers, printed text, and/or other visual displays that are permanently or temporarily affixed to the packaging of such food products.

The food product 520 may alternatively comprise fresh or unpackaged food items such as fruits, vegetables, meats, seafood, bakery items, and/or bulk goods that may not have ingredient information directly attached to the individual items. In such cases, the ingredient data capture module 506 may capture ingredient data from associated information sources that are positioned near, displayed with, or otherwise linked to the food product 520. These associated information sources may include shelf tags, display signs, digital screens, printed cards, and/or other informational materials that provide ingredient details for the corresponding food products.

In some embodiments, the food product 520 may be associated with machine-readable objects that contain or reference ingredient information but are not physically attached to the food product itself. These machine-readable objects may include QR codes posted on store shelves, NFC tags embedded in display cases, RFID tags attached to storage containers, BLE beacons positioned in product areas, and/or barcode labels placed on bins or dispensers containing bulk food items. The ingredient data capture module 506 may detect and process these machine-readable objects to obtain ingredient information for the associated food products, even when the objects are spatially separated from the actual food items.

The ingredient data capture module 506 may also capture ingredient data from temporary or removable information sources associated with the food product 520. These sources may include removable stickers or tags that can be detached from the food product, twist ties or clips that contain printed ingredient information, or separate information cards that accompany the food product during distribution or sale. In restaurant or food service environments, the food product 520 may be associated with menu descriptions, ingredient disclosure statements, or allergen information displays that the ingredient data capture module 506 may process to obtain relevant ingredient data.

The ingredient data capture module 506 may implement food product identification capabilities that link captured ingredient data to specific food items, even when the ingredient information is obtained from sources that are not directly attached to the food product 520. In some cases, the module 506 may utilize visual recognition algorithms to identify the food product 520 based on its appearance, shape, color, or other visual characteristics, then associate this identification with ingredient data captured from nearby information sources. The ingredient data capture module 506 may employ location-based matching that correlates the physical position of the food product 520 with the location of associated information sources, ensuring accurate pairing of ingredient data with the correct food items.

In some embodiments, the captured ingredient data 522 may be obtained, in whole or in part, using direct API integration from manufacturers, suppliers, regulatory databases, third-party nutrition services, and/or other external data sources. The ingredient data capture module 506 may implement API connectors that establish secure communication channels with manufacturer databases to retrieve current ingredient formulations, nutritional information, and allergen declarations directly from the source. These API integrations may enable access to more comprehensive and up-to-date ingredient information than what may be available on physical packaging, particularly for products that have undergone recent formulation changes. The ingredient data capture module 506 may utilize standardized data exchange protocols such as REST, GraphQL, and/or SOAP to facilitate seamless information retrieval from multiple external sources simultaneously. In some cases, the module 506 may implement authentication mechanisms including OAuth, API keys, and/or digital certificates to ensure secure access to proprietary ingredient databases maintained by food manufacturers or industry consortiums.

Referring to FIG. 4, the method 400 continues with operation 406, which involves deriving ingredient information from the captured ingredient data, the ingredient information representing a plurality of identified ingredients. As shown in FIG. 5, this operation may be performed by the ingredient information derivation module 508 of the personalized ingredient analysis system 500. The ingredient information derivation module 508 may process the captured ingredient data 522 received from the ingredient data capture module 506 to generate derived ingredient information 524 that represents a plurality of identified ingredients for subsequent analysis. In some embodiments, deriving the ingredient information may involve multiple processing stages, including text extraction, tokenization, ingredient identification, and/or descriptor association, which may work together to transform raw captured data into structured ingredient information suitable for health analysis.

In some embodiments, deriving the ingredient information may comprise performing optical character recognition (OCR) processing on captured image data to extract text. The ingredient information derivation module 508 may employ OCR algorithms to analyze image data captured by the image capture device, converting visual text elements from ingredient labels into machine-readable text format. For example, when the captured ingredient data 522 includes a photograph of an ingredient list on food product 520 packaging, the ingredient information derivation module 508 may apply OCR processing to identify and extract individual ingredient names, preservatives, additives, and nutritional components from the visual text. The OCR processing may handle various font types, sizes, and formatting styles commonly found on food packaging, ensuring accurate text extraction across different product types and manufacturers. Once the text is extracted through OCR processing, the ingredient information derivation module 508 may proceed to parse and structure the extracted text to identify individual ingredients and their associated descriptors.

The ingredient information derivation module 508 may implement tokenization processes to break down extracted text into discrete, analyzable units. In some cases, the tokenization process may involve parsing the extracted text to identify individual components (e.g., words, phrases, and/or punctuation marks) that can be processed as separate tokens. The ingredient information derivation module 508 may utilize various tokenization techniques, including whitespace tokenization that separates text based on spaces and line breaks, punctuation-based tokenization that uses commas, semicolons, and parentheses as delimiters, and/or pattern-based tokenization that recognizes ingredient list formatting conventions such as numbered lists or bullet points. For example, when processing extracted text such as “Water, Sugar (Cane Sugar), Salt, Natural Flavors,” the tokenization process may generate individual tokens including “Water,” “Sugar,” “Cane Sugar,” “Salt,” and “Natural Flavors.” The ingredient information derivation module 508 may implement advanced tokenization algorithms that recognize compound ingredient names, chemical nomenclature, and ingredient modifiers, ensuring that complex ingredient descriptions are properly segmented for subsequent analysis.

The ingredient information derivation module 508 may implement a multi-pass OCR processing system that combines multiple OCR engines for enhanced accuracy. In some cases, the module 508 may utilize a primary OCR engine such as Google Vision API to process the captured image data, followed by a secondary OCR engine such as Tesseract to analyze regions that were not successfully recognized in the initial pass. The ingredient information derivation module 508 may detect unrecognized regions by analyzing confidence scores from the primary OCR output and applying the secondary OCR engine specifically to these problematic areas. The module 508 may further implement error correction algorithms that compare extracted text against predefined ingredient dictionaries, applying Levenshtein distance calculations to identify and correct OCR errors where the distance between an extracted word and a known ingredient term is below a predetermined threshold. These error correction processes may work in conjunction with the tokenization and ingredient identification processes described above, ensuring that OCR errors do not propagate through the ingredient parsing pipeline.

The ingredient information derivation module 508 may incorporate image preprocessing capabilities to optimize text extraction accuracy before applying tokenization and ingredient identification processes. In some cases, the module 508 may apply histogram equalization to balance image contrast, bilateral denoising filters to smooth noise while preserving text edges, and perspective correction algorithms that detect label edges using Hough Transform techniques and warp distorted images to planar views. The preprocessing pipeline may enable accurate text extraction from challenging packaging formats, including curved labels, multi-column ingredient lists, and low-contrast printing commonly found on food packaging. The ingredient information derivation module 508 may perform connected component analysis to segment captured images into distinct text blocks, tables, and graphical elements. The module 508 may route different image regions to specialized OCR engines optimized for specific content types. For example, tabular ingredient lists may be processed by OCR engines configured for structured grid-based text extraction with column/row detection, while paragraph-format ingredient descriptions may be routed to OCR engines optimized for continuous text flow and natural language processing. Each specialized OCR engine may implement distinct preprocessing filters, recognition models, and/or post-processing rules tailored to the characteristics of its target content format.

In some embodiments, the module 508 may implement semantic segmentation algorithms that distinguish ingredient text from marketing claims, brand names, and nutritional information panels, ensuring that only relevant ingredient data is extracted for subsequent tokenization and analysis. The ingredient information derivation module 508 may apply specialized parsing rules for different types of ingredient list formats, including comma-separated lists, bulleted ingredient lists, and tabular ingredient presentations. The module 508 may implement content-type detection algorithms that analyze layout patterns, text density, and structural markers to automatically classify regions as tabular or paragraph format. Based on this classification, the module 508 may dynamically select and configure appropriate OCR processing pipelines. For example, grid-based segmentation and table structure recognition may be used for tabular regions, while continuous text flow models may be applied to paragraph regions. The module 508 may also implement context-aware tokenization that recognizes ingredient list boundaries and separates ingredient information from other product information such as nutritional facts, allergen warnings, or preparation instructions. These segmentation and routing capabilities may work in conjunction with the specialized OCR engines to ensure optimal text extraction and tokenization for each content format.

The ingredient information derivation module 508 may derive the ingredient information based on a QR code in the captured image data. In some cases, the food product 520 may include QR codes that contain encoded ingredient information, and the ingredient information derivation module 508 may decode these QR codes to obtain comprehensive ingredient details. The QR code processing may provide access to detailed ingredient databases maintained by manufacturers, potentially including information beyond what is visible on physical packaging labels.

The ingredient information derivation module 508 may derive the ingredient information based on a scanned barcode, utilizing barcode data captured by the barcode scanner to retrieve ingredient information from product databases and/or manufacturer systems.

The ingredient information derivation module 508 may calculate word-level and character-level confidence scores for extracted text, flagging low-confidence extractions for manual verification or recapture prompts. In some cases, when confidence scores fall below predetermined thresholds, the module 508 may automatically trigger alternative capture methods or request user confirmation of uncertain ingredient identifications. The confidence scoring system may contribute to overall system reliability by ensuring that only high-quality ingredient data proceeds to subsequent analysis stages. The ingredient information derivation module 508 may implement intelligent selection logic that determines the optimal derivation method based on available data sources and capture quality.

In some embodiments, the module 508 may prioritize barcode scanning for rapid structured data retrieval, fall back to QR code processing when barcodes are unavailable or unreadable, and utilize OCR processing as a final option when machine-readable codes are not present. The module 508 may also cross-validate ingredient information obtained through different methods, comparing OCR-extracted ingredient lists with database-retrieved information to identify discrepancies and ensure data accuracy.

The ingredient information derivation module 508 may derive the ingredient information based on a read RFID tag identifier. When the captured ingredient data 522 includes RFID tag information from the food product 520, the ingredient information derivation module 508 may process the RFID tag identifier to access stored ingredient data associated with the specific product. The RFID-based derivation may enable access to real-time ingredient information that may be updated by manufacturers or suppliers, potentially providing more current data than static packaging labels. The ingredient information derivation module 508 may access multiple product databases simultaneously, including manufacturer databases, regulatory databases such as OpenFDA, and third-party ingredient repositories.

In some cases, the module 508 may implement caching mechanisms that store frequently accessed ingredient information locally while maintaining synchronization with remote databases through scheduled updates. The real-time integration capability may enable access to the most current ingredient formulations and regulatory status information, potentially identifying recent formula changes or safety alerts that may not be reflected on physical packaging labels. The ingredient information derivation module 508 may implement multi-tiered caching with refresh intervals tailored to data volatility. For example, allergen classifications may be updated daily, regulatory status may be updated weekly, and research findings may be updated monthly. The module 508 may employ differential synchronization that only downloads changed records to minimize bandwidth usage while maintaining data currency. In some embodiments, the module 508 may implement priority-based caching where critical safety data receives more frequent updates than general nutritional information. The caching system may maintain separate update schedules for different regulatory jurisdictions to align with their respective publication cadences.

The ingredient information derivation module 508 may implement multiple derivation approaches simultaneously or in sequence, selecting the most appropriate method based on the type of captured ingredient data 522 available and the specific characteristics of the food product 520 being analyzed.

Referring to FIG. 4, the method 400 continues with operation 408, which involves analyzing the derived ingredient information 524 using an AI analysis module 510 to determine health implications, wherein the AI analysis module 510 comprises at least one of a natural language processing model or a machine learning model trained on nutritional and health data. As shown in FIG. 5, this operation may be performed by the AI analysis module 510 of the personalized ingredient analysis system 500. The AI analysis module 510 may process the derived ingredient information 524 received from the ingredient information derivation module 508 to generate health implications 526 that characterize potential health impacts associated with the plurality of identified ingredients.

In some embodiments, the AI analysis module 510 may utilize a natural language processing model to analyze the derived ingredient information 524 and determine health implications for each identified ingredient. The natural language processing model may process ingredient names, chemical compounds, and/or additive descriptions to extract semantic meaning and contextual health information. For example, when the derived ingredient information 524 includes ingredients such as “sodium benzoate” or “high fructose corn syrup,” the natural language processing model may identify these substances and associate them with relevant health implications based on nutritional and medical literature. The AI analysis module 510 may apply natural language understanding techniques to interpret complex ingredient descriptions, including chemical names, alternative ingredient designations, and compound ingredient formulations.

The AI analysis module 510 may alternatively or additionally employ a machine learning model trained on nutritional and health data to determine health implications from the derived ingredient information 524. In some cases, the machine learning model may comprise supervised learning algorithms trained on datasets that correlate specific ingredients with documented health effects, allergen information, and nutritional impacts. The machine learning model may analyze patterns in ingredient combinations, dosage levels, and interaction effects to generate comprehensive health implications 526 for the food product 520. Additionally, the AI analysis module 510 may utilize ensemble methods that combine multiple machine learning models to improve accuracy and reliability of health implication determinations.

The AI analysis module 510 may implement deep learning architectures, such as transformer-based models or neural networks, to process complex ingredient relationships and generate nuanced health implications 526. The artificial intelligence system may analyze not only individual ingredients but also potential synergistic effects between multiple ingredients present in the food product 520.

For example, the AI analysis module 510 may identify that certain preservatives may have amplified effects when combined with specific artificial colors or that particular ingredient combinations may pose heightened risks for individuals with certain health conditions. The AI analysis module 510 may implement real-time database integration techniques to cross-reference ingredient data against multiple authoritative health databases simultaneously, such as FDA GRAS databases, NIH compound databases, and international regulatory databases including EFSA or Health Canada databases.

The AI analysis module 510 may employ API integration frameworks that enable dynamic retrieval of updated regulatory status information, ensuring that health implications 526 reflect current scientific understanding and regulatory classifications. The health implications 526 generated by the AI analysis module 510 may include categorized risk assessments, potential allergen warnings, nutritional impact summaries, and interaction alerts that provide comprehensive analysis of the derived ingredient information 524 for subsequent comparison against user health profiles. In some embodiments, the AI analysis module 510 may calculate an overall risk score for the food product 520 based on risk scores assigned to individual ingredients within the plurality of identified ingredients, wherein the overall risk score may be computed, for example, using weighted aggregation algorithms that consider the relative severity and importance of individual ingredient risks within the context of the user health profile data 518.

The AI analysis module 510 may utilize ontological reasoning systems that leverage structured knowledge representations of ingredient relationships, chemical taxonomies, and health condition hierarchies. These ontology-based approaches may enable the AI analysis module 510 to infer implicit relationships between ingredients and health conditions that may not be explicitly documented in training data. For example, the AI analysis module 510 may use chemical ontologies to identify that an ingredient belongs to a broader class of compounds known to affect specific physiological systems. The AI analysis module 510 may implement probabilistic analysis techniques that quantify uncertainty in health implication determinations, including Bayesian inference models that incorporate prior knowledge about ingredient safety with observed data to generate confidence intervals for risk assessments.

In some cases, the AI analysis module 510 may employ Monte Carlo simulation techniques to model variability in ingredient concentrations and their potential health impacts across different population groups. The AI analysis module 510 may incorporate temporal analysis capabilities that track changes in ingredient safety profiles over time based on emerging research and regulatory updates, implementing time-series analysis techniques to identify trends in adverse event reporting or evolving scientific consensus regarding specific ingredients.

The AI analysis module 510 may employ multi-modal analysis techniques that integrate textual ingredient information with other data types, such as molecular structure data, toxicological study results, or epidemiological data, enabling more comprehensive health implication assessments by considering multiple dimensions of ingredient characteristics simultaneously.

In addition to artificial intelligence techniques, the AI analysis module 510 may employ various non-AI techniques to analyze the derived ingredient information 524 and determine health implications. For example, the AI analysis module 510 may utilize a rule-based expert system that applies predefined if-then rules to ingredient data, such as flagging specific E-numbers or additives based on regulatory classifications.

The AI analysis module 510 may implement statistical analysis methods, including correlation analysis, regression models, and hypothesis testing to identify relationships between ingredients and health outcomes without requiring machine learning algorithms. In some embodiments, the AI analysis module 510 may incorporate deterministic lookup tables that map ingredient names directly to known health implications based on established medical literature, regulatory databases, or nutritional guidelines.

The AI analysis module 510 may employ Boolean logic operations to evaluate ingredient combinations against predefined health criteria, such as checking for the presence of common allergens or restricted substances in specific dietary protocols. The AI analysis module 510 may implement heuristic algorithms that apply experience-based techniques to solve ingredient analysis problems without requiring complex AI models.

These heuristic approaches may include pattern matching against known ingredient synonyms, threshold-based classification of ingredient concentrations, and decision tree frameworks for categorizing ingredients by health impact. The AI analysis module 510 may utilize graph-based analysis to map ingredient relationships and identify potential interaction pathways between components, leveraging established biochemical knowledge without machine learning.

In some cases, the AI analysis module 510 may employ simple string matching and regular expressions to identify ingredient patterns that correspond to known health concerns, such as detecting variations of glutamate compounds for individuals with sensitivities. These non-AI techniques may operate independently or in conjunction with the AI-based methods to provide comprehensive health implications 526 that incorporate multiple analytical approaches.

The health implications 526 generated by the AI analysis module 510 may encompass a wide range of health-related information that facilitates comprehensive comparison with the user health profile data 518. In some embodiments, the health implications 526 may include allergen identification and severity assessments that categorize ingredients according to their potential to trigger allergic reactions. For example, the AI analysis module 510 may identify ingredients such as wheat proteins, milk derivatives, soy lecithin, and/or tree nut oils and associate these substances with specific allergen categories recognized by regulatory agencies such as the FDA's major food allergen list. The health implications 526 may further include cross-reactivity information that identifies ingredients that may trigger reactions in individuals with related allergies, such as identifying lupin flour as a potential concern for individuals with peanut allergies.

The AI analysis module 510 may generate health implications 526 that include dietary restriction compatibility assessments for various lifestyle and medical dietary requirements. In some cases, the health implications 526 may categorize ingredients according to their compatibility with vegetarian, vegan, kosher, halal, gluten-free, dairy-free, and/or ketogenic dietary patterns. The AI analysis module 510 may identify ingredients such as gelatin, carmine, and/or rennet that may conflict with vegetarian or vegan preferences, or ingredients like modified food starch that may contain gluten and pose concerns for individuals with celiac disease. The health implications 526 may include information about ingredient processing methods that affect dietary compatibility, such as identifying whether sugar has been processed using bone char or whether wine has been clarified using animal-derived fining agents.

With continued reference to FIG. 5, the health implications 526 may include medical condition-specific risk assessments that evaluate ingredient compatibility with various health conditions documented in the user health profile data 518. The AI analysis module 510 may generate implications related to cardiovascular health, identifying ingredients such as trans fats, high sodium content, and/or excessive saturated fats that may pose risks for individuals with hypertension, heart disease, and/or hyperlipidemia. For diabetes management, the health implications 526 may include glycemic impact assessments that evaluate ingredients such as high fructose corn syrup, dextrose, and/or other rapidly absorbed carbohydrates that may cause blood glucose spikes. The AI analysis module 510 may identify ingredients that may interact with diabetes medications, such as chromium supplements that may enhance insulin sensitivity or artificial sweeteners that may affect glucose metabolism.

The health implications 526 may include kidney function considerations that identify ingredients potentially problematic for individuals with chronic kidney disease or other renal conditions. In some embodiments, the AI analysis module 510 may flag ingredients with high phosphorus content, such as phosphoric acid or sodium phosphates, that may contribute to mineral imbalances in individuals with compromised kidney function. The health implications 526 may identify ingredients with high potassium content, such as potassium chloride or cream of tartar, that may require monitoring in individuals taking certain medications or those with advanced kidney disease. The AI analysis module 510 may assess protein content and quality in ingredients, providing implications relevant to individuals who need to restrict or modify their protein intake due to kidney disease.

The AI analysis module 510 may generate health implications 526 that address gastrointestinal health concerns and digestive sensitivities. The health implications 526 may include assessments of ingredients that commonly trigger symptoms in individuals with irritable bowel syndrome, such as high-FODMAP ingredients including fructose, lactose, fructans, and/or sugar alcohols like sorbitol and mannitol. In some cases, the AI analysis module 510 may identify ingredients that may exacerbate inflammatory bowel conditions, such as carrageenan, artificial colors, and/or certain emulsifiers that have been associated with intestinal inflammation in research studies. The health implications 526 may include information about ingredients that may affect gut microbiome composition, such as prebiotics, probiotics, and/or antimicrobial preservatives that may alter beneficial bacterial populations.

The health implications 526 may encompass neurological and cognitive health considerations that evaluate ingredients for their potential effects on brain function and neurological conditions. The AI analysis module 510 may identify ingredients such as aspartame that may trigger headaches or migraines in sensitive individuals, or monosodium glutamate that may cause symptoms in individuals with MSG sensitivity. For individuals with attention deficit hyperactivity disorder or autism spectrum disorders, the health implications 526 may include assessments of artificial colors, preservatives, and/or flavor enhancers that have been associated with behavioral changes in some research studies. The AI analysis module 510 may also evaluate ingredients for their potential neuroprotective or neurotoxic properties, identifying compounds that may support or impair cognitive function.

The health implications 526 may include hormonal and endocrine system considerations that assess ingredients for their potential effects on hormone production, metabolism, and/or endocrine function. The AI analysis module 510 may identify ingredients such as soy isoflavones that may have estrogenic effects, or ingredients containing phytoestrogens that may be relevant for individuals with hormone-sensitive conditions such as breast cancer or endometriosis. The health implications 526 may include assessments of ingredients that may affect thyroid function, such as soy products that may interfere with thyroid hormone absorption or iodine-containing ingredients that may be problematic for individuals with thyroid disorders. Additionally, the AI analysis module 510 may evaluate ingredients for their potential effects on blood sugar regulation and insulin sensitivity, identifying compounds that may support or impair metabolic health.

The AI analysis module 510 may generate health implications 526 that address medication interactions and contraindications with pharmaceutical treatments. In some embodiments, the health implications 526 may identify ingredients that may enhance or inhibit the absorption, metabolism, and/or effectiveness of medications listed in the user health profile data 518. For example, the AI analysis module 510 may flag ingredients high in vitamin K, such as leafy green extracts, that may interfere with warfarin therapy, or identify calcium-rich ingredients that may reduce the absorption of certain antibiotics or thyroid medications. The health implications 526 may also include information about ingredients that may potentiate medication side effects, such as identifying tyramine-containing ingredients that may be problematic for individuals taking monoamine oxidase inhibitors.

The health implications 526 may include age-specific and life stage considerations that evaluate ingredient safety and appropriateness for different demographic groups. The AI analysis module 510 may identify ingredients that may be inappropriate for pregnant or breastfeeding women, such as high levels of caffeine, artificial sweeteners like saccharin, and/or herbs with potential teratogenic effects. For elderly individuals, the health implications 526 may include assessments of ingredients that may affect medication absorption, contribute to dehydration, and/or pose choking hazards due to texture modifications. The AI analysis module 510 may evaluate ingredients for their appropriateness in pediatric populations, identifying compounds that may affect growth, development, and/or behavior in children.

The AI analysis module 510 may generate health implications 526 that encompass environmental and occupational health considerations relevant to individuals with specific sensitivities or exposures. The health implications 526 may include assessments of ingredients that may contain or be contaminated with heavy metals, pesticide residues, and/or industrial chemicals that may pose cumulative health risks. In some cases, the AI analysis module 510 may identify ingredients derived from genetically modified organisms and provide implications relevant to individuals who prefer to avoid such ingredients for health or ethical reasons. The health implications 526 may also include information about ingredient processing methods that may introduce or remove beneficial or harmful compounds, such as hydrogenation processes that create trans fats or fermentation processes that may reduce antinutrient content.

Referring to FIG. 4, the method 400 continues with operation 410, which involves comparing the health implications against the user health profile data to produce comparison output. As shown in FIG. 5, this operation may be performed by the health implication comparison module 512 of the personalized ingredient analysis system 500. The health implication comparison module 512 may process the health implications 526 received from the AI analysis module 510 and compare these health implications against the user health profile data 518 to generate comparison results 528 that indicate compatibility between the identified ingredients and the user's specific health requirements.

In some embodiments, the health implication comparison module 512 may perform matching of each identified ingredient against specific elements within the user health profile data 518. The comparison process may evaluate whether individual ingredients or their associated health implications conflict with dietary restrictions, allergies, and/or health conditions stored in the user health profile data 518. For example, when the health implications 526 indicate that a particular ingredient contains gluten, the health implication comparison module 512 may check the user health profile data 518 to determine whether the user 502 has specified gluten intolerance or celiac disease as a health condition.

The health implication comparison module 512 may flag potential conflicts and generate alerts when ingredients pose risks based on the user's documented health profile. The module 512 may implement threshold-based sensitivity matching that utilizes quantitative limits stored in the user health profile data 518, such as parts-per-million (ppm) thresholds for specific allergens. In some cases, the module 512 may store per-allergy ppm limits, such as sulfites greater than 10 ppm triggering a flag, or gluten concentrations above 20 ppm indicating incompatibility for users with celiac disease. The threshold-based matching may enable precise evaluation of ingredient concentrations against individual tolerance levels rather than binary presence-absence determinations. These threshold values and tolerance levels may differ between different users 502, reflecting the personalized nature of individual health conditions and sensitivities that require customized assessment parameters for accurate compatibility evaluation.

The health implication comparison module 512 may implement weighted scoring algorithms that assign different priority levels to various types of health implications based on the severity and specificity of conditions in the user health profile data 518. In some cases, the module 512 may treat allergen-related implications with higher priority than general nutritional concerns, ensuring that potentially dangerous ingredient interactions receive appropriate attention during the comparison process. The health implication comparison module 512 may categorize sensitivity levels into discrete categories, such as low sensitivity for minor dietary preferences, medium sensitivity for moderate health concerns or food intolerances, and high sensitivity for severe allergies or critical health conditions that may pose immediate health risks. These sensitivity level categories may vary between different users 502, as each individual user may have unique health conditions, tolerance thresholds, and risk profiles that require personalized assessment criteria. These sensitivity level categories may enable the module 512 to apply appropriate weighting and prioritization during the comparison process, ensuring that high sensitivity conditions receive the most stringent evaluation criteria.

The health implication comparison module 512 may analyze cumulative effects of multiple ingredients, considering how combinations of substances might interact with the user's health conditions, dietary restrictions, and/or medication schedules. The module 512 may employ pattern-matching algorithms that utilize regular expressions and embedding-based similarity scoring to identify ingredient variants and synonyms. For example, the module 512 may implement cosine similarity calculations between embedded ingredient terms and ontology terms, flagging matches when similarity scores exceed predetermined thresholds such as 0.85.

The health implication comparison module 512 may incorporate ontology-based lookup systems that utilize graph databases such as Neo4j to store and query ingredient relationships. In some embodiments, the module 512 may execute SPARQL queries against food ontology databases to identify canonical ingredient names and their associated health implications. The module 512 may incorporate temporal factors, such as medication schedules, dietary timing requirements, and/or seasonal allergies specified in the user health profile data 518, when evaluating ingredient compatibility.

The health implication comparison module 512 may implement multi-pass validation against external health databases with conflict resolution logic to ensure accuracy and consistency of comparison results 528. In some cases, the module 512 may cross-reference ingredient assessments against multiple authoritative sources including NIH, Mayo Clinic, OpenFDA, and EFSA databases through real-time API integration. When conflicting information is retrieved from different sources, the module 512 may apply conflict resolution algorithms that prioritize higher severity ratings or flag ingredients for manual review.

The health implication comparison module 512 may integrate adaptive learning mechanisms that incorporate user feedback to refine future comparison processes. For example, when users override system recommendations or flag false positives, the module 512 may adjust sensitivity thresholds and matching criteria for similar ingredients in subsequent analyses. The module 512 may implement confidence scoring frameworks that calculate geometric means of multiple confidence factors, including OCR confidence scores, AI analysis confidence scores, and data source reliability scores. In some embodiments, the confidence scoring may influence the comparison process by adjusting threshold values or requiring additional validation for low-confidence assessments.

The uncertainty quantification framework may incorporate measures of agreement between multiple artificial intelligence models used in analyzing the ingredient information, wherein disagreement between models may reduce the overall confidence score while consensus may increase confidence levels. The confidence scoring framework may enable modification of the visual Go/No-Go indicator based on calculated confidence scores, such as displaying uncertainty indicators, confidence percentages, or modified visual elements when confidence levels fall below predetermined thresholds.

The health implication comparison module 512 may implement confidence-gated UI control flow mechanisms that modify the user interface based on calculated confidence scores. In some embodiments, when the overall confidence score falls below a predetermined threshold, the health implication comparison module 512 may suppress the display of visual Go/No-Go indicators and instead trigger alternative user interface states. For example, when confidence scores fall below a threshold value such as 0.7, 0.75, 0.8, or 0.85, the module 512 may generate UI control signals that prevent the output generation module 514 from displaying definitive compatibility assessments. The confidence-gated control flow may include automatic recapture prompts that request users to capture additional and/or clearer images of ingredient labels, switch to alternative capture modalities such as barcode scanning when OCR confidence is insufficient, and/or manually verify uncertain ingredient identifications through text input interfaces.

The health implication comparison module 512 may implement specific UI state transitions based on confidence thresholds, where different confidence ranges trigger distinct user interface behaviors. In some cases, confidence scores above a high threshold may enable full Go/No-Go indicator display with detailed ingredient breakdowns, while confidence scores in intermediate ranges may display partial results with uncertainty warnings and options for additional verification. When confidence scores fall below critical thresholds, the module 512 may completely suppress compatibility indicators and instead display messages such as “Insufficient confidence-please recapture image” or “Unable to analyze-try barcode scanning.” The confidence-gated control flow may also implement progressive disclosure mechanisms where low-confidence ingredients are flagged for manual review while high-confidence ingredients proceed through automated analysis, enabling users to focus verification efforts on uncertain elements while maintaining system efficiency for reliable detections.

The health implication comparison module 512 may generate detailed comparison results 528 that categorize each ingredient according to its compatibility with the user health profile data 518. The comparison results 528 may include risk classifications, compatibility scores, confidence metrics, and/or specific explanations for why certain ingredients may or may not align with the user's health requirements. The health implication comparison module 512 may also identify alternative ingredients or suggest modifications that could improve overall product compatibility with the user health profile data 518.

The comparison output produced by the health implication comparison module 512 may serve as the foundation for subsequent decision-making processes that generate personalized recommendations and visual indicators for the user 502.

Referring to FIG. 4, the method 400 continues with operation 412, which involves generating an output based on the comparison output, wherein the output includes a visual Go/No-Go indicator representing compatibility between the user health profile data and at least one of the food product or at least one of the plurality of identified ingredients. As shown in FIG. 5, this operation may be performed by the output generation module 514 of the personalized ingredient analysis system 500. The output generation module 514 may process the comparison results 528 received from the health implication comparison module 512 to create generated output 530 that provides clear, actionable guidance to the user 502 regarding the suitability of the food product 520 based on their individual health profile.

In some embodiments, the output generation module 514 may apply a weighted scoring system to the determined health implications, wherein different types of determined health implications are assigned different weights. For example, the weighted scoring system may assign a first weight to allergen-related implications, a second weight to additive-related implications, and a third weight to preference-related implications, wherein the first weight is greater than the second weight, and wherein the second weight is greater than the third weight. This hierarchical weighting approach may ensure, for example, that life-threatening allergic reactions receive the highest priority in the output generation process, followed by potentially harmful additives, and finally by personal dietary preferences that may not pose immediate health risks.

The visual Go/No-Go indicator may operate at any of a variety of granularity levels, such as by providing compatibility assessments for any one or more of the following: individual ingredients, groups of ingredients, or the food product 520 as a whole. As this implies the generated output 530 may include one or more outputs (e.g., one or more visual Go/No-Go indicators, such as a plurality of visual Go/No-Go indicators, each corresponding to a distinct food ingredient within the food product).

In some embodiments, the output generation module 514 may generate the visual Go/No-Go indicator by evaluating the overall compatibility between the user health profile data 518 and the food product 520 based on the comparison results 528. The visual Go/No-Go indicator may comprise graphical elements, color-coded signals, and/or symbolic representations that immediately convey whether the food product 520 aligns with the user's health requirements. For example, the output generation module 514 may display a green “Go” indicator when the comparison results 528 indicate that all identified ingredients are compatible with the user health profile data 518, or a red “No-Go” indicator when one or more ingredients pose potential risks or conflicts. The visual indicator may include intermediate states, such as a yellow “Caution” signal, when certain ingredients require attention but do not represent immediate health concerns. This product-level assessment may provide users with a quick overall evaluation of the food product 520 without requiring detailed review of individual ingredient compatibility.

The output generation module 514 may alternatively or additionally generate the visual Go/No-Go indicator to represent compatibility with at least one of the plurality of identified ingredients rather than the food product 520 as a whole. In some cases, the generated output 530 may include individual compatibility indicators for each ingredient identified in the derived ingredient information 524, allowing the user 502 to understand which specific components of the food product 520 align or conflict with their health profile. For example, the output generation module 514 may display a list where each ingredient is accompanied by its own color-coded indicator, such as “Wheat flour: No-Go (gluten allergy),” “Sugar: Caution (diabetes management),” and/or “Salt: Go (within acceptable limits).” The output generation module 514 may implement hierarchical display systems that show both overall product compatibility and ingredient-level compatibility assessments, providing comprehensive guidance for informed decision-making. In some embodiments, ingredient-level indicators may be aggregated using weighted algorithms to determine the overall product-level Go/No-Go status, where critical allergens may override otherwise acceptable ingredients.

The output generation module 514 may calculate a weighted score based on the determined health implications, calculate a confidence score for the weighted score, and generate the output based on both the weighted score and the confidence score. In some cases, the output generation module 514 may adjust the weighted score based on the confidence score to produce a final compatibility score for the food product 520, wherein lower confidence scores may result in more conservative compatibility assessments to ensure user safety.

The output generation module 514 may incorporate additional information elements into the generated output 530 beyond the visual Go/No-Go indicator. The generated output 530 may encompass various forms of output content and presentation formats to accommodate different user needs and device capabilities, with the flexibility to present Go/No-Go assessments at both product and ingredient levels simultaneously or selectively. In some embodiments, the generated output 530 may include textual content such as detailed explanations of why certain ingredients received specific compatibility ratings, plain-language descriptions of ingredient health implications, alternative product suggestions that better align with the user health profile data 518, and/or educational information about particular ingredients and their regulatory status. The generated output 530 may include numerical data such as confidence scores that indicate the reliability of the compatibility assessments, risk percentages associated with specific ingredients, and/or quantitative measures of ingredient concentrations relative to safety thresholds, helping the user 502 understand the certainty level and severity associated with the recommendations at both individual ingredient and overall product levels.

The generated output 530 may include interactive elements that enable user engagement with the analysis results, such as expandable sections for detailed ingredient information, clickable links to authoritative health sources, user rating systems for feedback collection, and/or customizable display preferences for information prioritization. Users may interact with ingredient-level Go/No-Go indicators to access detailed explanations, while product-level indicators may provide summary views with options to drill down into constituent ingredient assessments. In some cases, the generated output 530 may comprise multimedia content including audio narration of ingredient warnings for accessibility purposes, video explanations of health implications, graphical charts showing ingredient risk levels over time, and/or animated demonstrations of potential health effects. The output generation module 514 may also generate structured data formats such as exportable reports for healthcare providers, machine-readable data for integration with other health applications, standardized allergen alerts compatible with medical record systems, and/or formatted summaries suitable for printing or sharing with family members, with each format capable of presenting Go/No-Go assessments at the appropriate granularity level for the intended use case.

The generated output 530 may be formatted for optimal display across various presentation modalities and computing environments, with the visual Go/No-Go indicator maintaining clarity and functionality whether presented at the product level, ingredient level, or both simultaneously. For example, the output generation module 514 may format the generated output 530 as mobile application interfaces with touch-responsive elements that allow users to toggle between product-level and ingredient-level views, web-based dashboards with real-time data updates showing hierarchical compatibility assessments, voice-activated responses for hands-free operation that can announce both overall product status and specific ingredient concerns, and/or augmented reality overlays that display ingredient information directly on product packaging with individual ingredient indicators visible alongside overall product assessment.

The generated output 530 may encompass notification systems such as push notifications for ingredient alerts that specify whether the alert applies to individual ingredients or the entire product, email summaries of scanning sessions with detailed breakdowns of both product and ingredient assessments, text message warnings for high-risk ingredients with clear indication of scope, and/or calendar reminders for dietary compliance monitoring.

In some embodiments, the generated output 530 may include contextual information such as location-based ingredient availability, seasonal allergy considerations, cultural dietary preferences, and/or time-sensitive health recommendations, ensuring that the visual Go/No-Go indicator and accompanying information remain clear and accessible across different screen sizes, interface configurations, and user contexts, regardless of whether the assessment is presented at the product level, ingredient level, or both.

Referring to FIG. 4, the method 400 concludes with operation 414, which involves providing the output to a user. As shown in FIG. 5, this operation may be performed by the user output provision module 516 of the personalized ingredient analysis system 500. The user output provision module 516 may manifest the comparison results 528 as provided output 532 that delivers the personalized ingredient analysis results to the user 502 through various output modalities. While FIG. 4 shows operation 412 (generating output) and operation 414 (providing output) as distinct operations for clarity, in some embodiments, these operations may be combined into a single operation that both generates and provides the output to the user 502.

As used herein, the term “manifesting” may refer to providing output to the user in any form, such as visual, auditory, and/or haptic output. The provided output 532 may represent an example of “manifested output” or a “manifestation” of the comparison results 528, where the analysis results are presented to the user 502 in a perceivable format that facilitates understanding and decision-making regarding the food product 520. In some embodiments, the user output provision module 516 may directly transform the comparison results 528 into the provided output 532 without requiring an intermediate generated output 530 as shown in FIG. 5.

In some embodiments, the user output provision module 516 may manifest the output through visual display mechanisms that present the visual Go/No-Go indicator and accompanying information on a screen or display device associated with the computing device. The visual manifestation may include graphical user interface elements that organize the compatibility assessments, ingredient analyses, and health implications in an intuitive layout that enables rapid comprehension by the user 502. For example, the user output provision module 516 may display the visual Go/No-Go indicator prominently at the top of the interface, followed by detailed ingredient breakdowns, risk assessments, and explanatory text that supports the overall compatibility determination. The user output provision module 516 may generate these visual elements directly from the comparison results 528 as part of the providing operation 414.

The user output provision module 516 may alternatively or additionally manifest the output through auditory output mechanisms that provide spoken summaries of the ingredient analysis results. In some cases, the auditory manifestation may include text-to-speech conversion of the compatibility assessments, allowing users with visual impairments or those in hands-free situations to receive the personalized ingredient analysis information. The user output provision module 516 may prioritize the most relevant information in the auditory output, such as allergen warnings or critical health conflicts, ensuring that users receive the most important guidance first when listening to the analysis results. This auditory manifestation may be generated directly from the comparison results 528 as part of operation 414, without requiring a separate generation step.

The user output provision module 516 may also manifest the output through haptic feedback mechanisms that provide tactile sensations to convey compatibility information. The haptic manifestation may include vibration patterns, force feedback, or other tactile signals that correspond to different compatibility levels indicated by the visual Go/No-Go indicator. For example, the user output provision module 516 may generate a gentle vibration for “Go” indicators, a pulsing vibration pattern for “Caution” signals, or a strong, rapid vibration sequence for “No-Go” warnings. The user output provision module 516 may combine multiple manifestation modalities simultaneously, providing visual, auditory, and haptic output concurrently to ensure that the provided output 532 reaches the user 502 through their preferred or most accessible sensory channels. In this way, operation 414 may encompass both the generation and provision of output to the user 502, thereby completing the personalized ingredient analysis process initiated by the method 400.

Embodiments of the present invention may be implemented using various deployment architectures that distribute processing capabilities between local computing devices and cloud-based systems. The personalized ingredient analysis system 500 may perform any combination of operations locally on the user 502's computing device, remotely in cloud-based infrastructure, or through hybrid architectures that leverage both local and remote processing capabilities. This flexible deployment approach may enable the system 500 to optimize performance, privacy, connectivity requirements, and computational resource utilization based on specific use cases and user preferences.

The ingredient data capture module 506 may operate primarily on local computing devices, such as smartphones or tablets, to leverage integrated hardware components including image capture devices, barcode scanners, and NFC readers. In some embodiments, the ingredient data capture module 506 may perform local image preprocessing operations such as contrast adjustment, noise reduction, and perspective correction to optimize captured ingredient data 522 before transmission to other system components. The module 506 may alternatively implement cloud-based image processing capabilities that utilize remote servers with specialized hardware accelerators for computationally intensive image enhancement operations. For example, the ingredient data capture module 506 may capture raw images locally on the user 502's mobile device, then transmit the captured images to cloud-based services that perform advanced image stabilization, lighting correction, and multi-frame super-resolution processing to improve text extraction accuracy.

Referring to FIG. 4, the ingredient information derivation module 508 may implement optical character recognition processing through various deployment configurations that balance accuracy, speed, and privacy considerations. In some cases, the ingredient information derivation module 508 may perform OCR processing entirely on the user 502's local device using on-device machine learning models optimized for mobile processors, enabling ingredient text extraction without requiring internet connectivity or data transmission to external servers. The module 508 may alternatively utilize cloud-based OCR services such as Google Vision API or Amazon Textract that provide enhanced accuracy and support for diverse languages and font types through powerful server-side processing capabilities. Hybrid implementations may combine local preprocessing with cloud-based OCR, where the ingredient information derivation module 508 performs initial text detection and region segmentation locally, then transmits only the relevant text regions to cloud services for detailed character recognition and text extraction.

The AI analysis module 510 may implement various deployment strategies for analyzing the derived ingredient information 524 and determining health implications 526. Local implementations may utilize on-device machine learning models that have been optimized for mobile deployment, such as quantized neural networks or knowledge-distilled models that provide ingredient analysis capabilities without requiring network connectivity. In some embodiments, the AI analysis module 510 may store compressed versions of nutritional databases and health implication rules locally on the user 502's device, enabling basic ingredient analysis and allergen detection in offline scenarios. Cloud-based implementations may leverage large language models and comprehensive health databases that exceed the storage and computational capabilities of mobile devices, providing more sophisticated analysis of ingredient interactions, medication contraindications, and personalized health recommendations. The AI analysis module 510 may implement federated learning approaches where local models on user devices contribute to collective learning while maintaining data privacy, with periodic model updates distributed from cloud infrastructure to improve analysis accuracy across the user base.

With continued reference to FIG. 5, the health implication comparison module 512 may perform user health profile matching through local processing to maintain privacy of sensitive health information, or through cloud-based systems that enable more comprehensive analysis and cross-referencing with external health databases. Local implementations may store the user health profile data 518 entirely on the user 502's device using encrypted storage mechanisms, with the health implication comparison module 512 performing compatibility assessments without transmitting personal health information to external systems. Cloud-based implementations may enable the health implication comparison module 512 to access real-time updates from authoritative health databases, regulatory agencies, and medical research institutions, providing more current and comprehensive health implications 526 for ingredient analysis. Hybrid approaches may perform initial compatibility screening locally using cached health profile data, then selectively query cloud services for detailed analysis of flagged ingredients or complex interaction assessments.

The output generation module 514 may implement local rendering capabilities that format and display the generated output 530 using the computing device's native user interface frameworks, ensuring responsive performance and offline accessibility. In some cases, the output generation module 514 may generate visual Go/No-Go indicators, detailed ingredient breakdowns, and explanatory content entirely on the user 502's device using locally stored templates and formatting rules. Cloud-based implementations may enable the output generation module 514 to access dynamic content generation services, real-time alternative product recommendations, and personalized educational materials that adapt based on current health trends and regulatory updates. The module 514 may implement progressive enhancement approaches where basic output generation occurs locally, with additional cloud-based services providing enhanced visualizations, multimedia content, and interactive elements when network connectivity is available.

The user output provision module 516 may manifest the provided output 532 through local device capabilities such as display screens, speakers, and haptic feedback systems, ensuring immediate responsiveness and accessibility regardless of network conditions. Local implementations may include text-to-speech synthesis, vibration pattern generation, and graphical rendering that operate independently of cloud services. In some embodiments, the user output provision module 516 may implement cloud-based accessibility services that provide enhanced audio descriptions, multi-language support, and adaptive interface modifications based on user accessibility preferences stored in cloud profiles. Hybrid implementations may combine local output rendering with cloud-based content delivery, where the user output provision module 516 displays immediate results locally while simultaneously retrieving enhanced content, alternative product suggestions, and updated health information from cloud services for subsequent presentation to the user 502.

The personalized ingredient analysis system 500 may implement intelligent workload distribution algorithms that automatically determine optimal deployment configurations based on factors such as network connectivity, device capabilities, battery life, privacy preferences, and processing requirements. In some cases, the system 500 may perform computationally intensive operations such as AI analysis and database queries in the cloud when high-speed connectivity is available, while falling back to local processing using cached data and simplified models when network access is limited or unavailable. When utilizing cloud-based processing for health-related data, the system 500 may employ HIPAA-compliant cloud services and maintain appropriate business associate agreements to ensure regulatory compliance for protected health information. The system 500 may implement data synchronization mechanisms that maintain consistency between local and cloud-based components, ensuring that user health profile updates, ingredient database changes, and analysis improvements are propagated across all deployment environments while respecting user privacy preferences and regulatory compliance requirements.

Embodiments of the present invention may provide several advantages over previous systems for nutritional analysis and ingredient assessment. Traditional ingredient analysis applications often rely on manual data entry and generic ingredient databases that fail to account for individual health profiles, dietary restrictions, and specific medical conditions. In contrast, embodiments of the personalized ingredient analysis system 500 may integrate multiple data capture modalities, including image capture devices, barcode scanners, and RFID tag readers, enabling rapid and accurate ingredient data acquisition without requiring tedious manual input from users. The AI analysis module 510 may leverage natural language processing models and machine learning algorithms trained on nutritional and health data to provide comprehensive health implications 526 that extend beyond simple ingredient identification to include potential allergen risks, medication interactions, and condition-specific dietary considerations.

Previous systems may provide one-size-fits-all recommendations that ignore individual health variations and fail to adapt to user-specific needs. Embodiments of the present invention may address these limitations through the health implication comparison module 512, which may compare determined health implications 526 against personalized user health profile data 518 to generate tailored compatibility assessments. The user health profile data 518 may encompass detailed information about dietary restrictions, allergies, health conditions, medication schedules, and individual tolerance thresholds, enabling the system 500 to provide personalized recommendations that account for the unique health requirements of each user 502. The weighted scoring algorithms implemented by the output generation module 514 may prioritize critical health factors such as severe allergies over general dietary preferences, ensuring that life-threatening risks receive appropriate emphasis in the visual Go/No-Go indicators.

Embodiments of the system 500 may offer enhanced accuracy and reliability compared to existing solutions through the integration of multiple confidence scoring mechanisms and uncertainty quantification frameworks. The confidence scoring system may combine OCR confidence scores from the ingredient information derivation module 508, AI analysis confidence scores from the AI analysis module 510, and data source reliability scores from external health databases to provide users with transparent assessments of recommendation certainty. Previous systems may lack such comprehensive confidence metrics, potentially leading to overconfidence in uncertain analyses or failure to alert users when additional verification may be warranted. The adaptive learning capabilities of embodiments of the present invention may enable continuous improvement of analysis accuracy through user feedback integration, allowing the system 500 to refine sensitivity thresholds and matching criteria based on real-world user experiences.

Embodiments of the present invention may provide superior data integration capabilities compared to conventional ingredient analysis systems. The AI analysis module 510 may access and cross-reference multiple authoritative health databases simultaneously, including FDA databases, NIH compound databases, Mayo Clinic resources, and international regulatory databases such as EFSA or Health Canada databases. This comprehensive data integration may enable more accurate and up-to-date health implications 526 compared to systems that rely on limited or outdated ingredient databases. The real-time API integration capabilities may ensure that regulatory status changes, safety alerts, and emerging research findings are incorporated into the analysis process, providing users with current information that may not be available through static database systems.

Embodiments of the present invention may offer enhanced accessibility and usability advantages over previous systems through multiple output modalities and presentation formats. The user output provision module 516 may manifest analysis results through visual, auditory, and haptic feedback mechanisms, accommodating users with different accessibility needs and usage contexts. Previous systems may rely primarily on visual displays that may be inadequate for users with visual impairments or those requiring hands-free operation. The hierarchical display systems implemented by the output generation module 514 may provide both product-level and ingredient-level compatibility assessments, enabling users to understand overall product suitability while also accessing detailed information about specific ingredients of concern. The interactive elements within the generated output 530 may allow users to drill down into detailed explanations, access authoritative health sources, and customize display preferences according to their individual information needs.

The multi-modal data capture capabilities of embodiments of the present invention may provide significant advantages over systems that rely solely on manual text input or basic barcode scanning. As shown in FIG. 5, the ingredient data capture module 506 may utilize image capture devices for OCR processing of ingredient labels, barcode scanners for rapid product identification, RFID tag readers for accessing embedded product information, voice recognition systems for hands-free data entry, and NFC readers for proximity-based data acquisition. This comprehensive approach may enable ingredient data capture in various environments and usage scenarios where single-modality systems may fail, such as when ingredient labels are damaged, barcodes are unreadable, or users have physical limitations that prevent certain interaction methods. The intelligent selection logic may automatically determine the optimal capture method based on available data sources and environmental conditions, providing seamless user experiences across different product types and scanning situations.

Embodiments of the present invention may provide enhanced personalization capabilities that extend beyond simple allergen detection to encompass comprehensive health profile matching. The user health profile data 518 may include quantitative thresholds for ingredient tolerances, medication interaction profiles, temporal dietary considerations, and life stage-specific nutritional requirements that enable more sophisticated compatibility assessments than binary presence-absence determinations used by previous systems. The sensitivity level categorization implemented by the health implication comparison module 512 may enable differentiated risk assessments that account for the varying severity of different health conditions and dietary restrictions. Previous systems may treat all dietary restrictions equally, potentially overwhelming users with unnecessary warnings while failing to adequately emphasize critical health risks that require immediate attention.

The modular architecture of embodiments of the present invention may enable adaptation across various application domains while maintaining the core technological framework. In some embodiments, the personalized ingredient analysis system may be configured for specialized applications including veterinary nutrition analysis for animal health management, cosmetic and skincare product evaluation, integration with electronic health record systems for clinical decision support, and humanitarian applications for disaster relief and food safety assessment. The system may also be implemented as an application programming interface for third-party integration into retail applications, smart appliances, and health technology platforms. Additionally, embodiments may include educational versions for nonprofit organizations and underserved populations, as well as specialized modules for supply chain transparency and regulatory compliance monitoring. These diverse applications may leverage the same fundamental components including the AI analysis module, health implication comparison capabilities, and adaptive learning mechanisms described herein, demonstrating the broad applicability of the core technological innovations.

Embodiments of the present invention include a variety of features that implement significantly more than an abstract idea. For example, embodiments of the personalized ingredient analysis system 500 may implement specific technological improvements to computer functionality through the integration of multiple specialized processing modules that work in coordination to solve technical problems in automated ingredient analysis and health assessment.

The ingredient information derivation module 508 may employ optical character recognition (OCR) processing techniques that combine multiple OCR engines with error correction algorithms to extract text from challenging packaging formats. In some cases, the module 508 may implement multi-pass OCR processing systems that utilize a primary OCR engine such as Google Vision API followed by a secondary OCR engine such as Tesseract to analyze regions with low confidence scores. The ingredient information derivation module 508 may apply image preprocessing capabilities including histogram equalization for contrast balancing, bilateral denoising filters for noise reduction while preserving text edges, and perspective correction algorithms that detect label edges using Hough Transform techniques. These technical implementations may address specific computational challenges in processing diverse packaging formats, curved labels, multi-column ingredient lists, and low-contrast printing commonly encountered in real-world food packaging scenarios.

The AI analysis module 510 may implement sophisticated machine learning architectures that process ingredient data through natural language processing models and machine learning models trained on nutritional and health data. In some embodiments, the AI analysis module 510 may utilize transformer-based models or neural networks to analyze complex ingredient relationships and generate nuanced health implications 526. The module 510 may employ ensemble methods that combine multiple machine learning models to improve accuracy and reliability of health implication determinations, addressing technical challenges in ingredient classification and health risk assessment that cannot be solved through conventional database lookup approaches. The AI analysis module 510 may implement ontological reasoning systems that leverage structured knowledge representations of ingredient relationships, chemical taxonomies, and health condition hierarchies to infer implicit relationships between ingredients and health conditions.

The health implication comparison module 512 may implement technical solutions for complex data matching and comparison operations that extend beyond simple keyword matching. The module 512 may employ pattern-matching algorithms that utilize regular expressions and embedding-based similarity scoring to identify ingredient variants and synonyms, calculating cosine similarity between embedded ingredient terms and ontology terms. In some cases, the health implication comparison module 512 may implement threshold-based sensitivity matching that utilizes quantitative limits stored in the user health profile data 518, such as parts-per-million thresholds for specific allergens. The module 512 may incorporate ontology-based lookup systems that utilize graph databases such as Neo4j to store and query ingredient relationships, executing SPARQL queries against food ontology databases to identify canonical ingredient names and their associated health implications.

The system 500 may implement multi-modal data integration techniques that combine information from diverse input sources including image capture devices, barcode scanners, RFID tag readers, voice recognition systems, and NFC readers. The ingredient data capture module 506 may employ intelligent selection logic that automatically determines optimal capture methods based on available data sources and environmental conditions, implementing fallback mechanisms when primary capture methods fail. This technical approach may solve computational problems related to data acquisition reliability and accuracy across varying environmental conditions and product packaging formats that cannot be addressed through single-modality systems.

Embodiments of the system 500 may implement confidence scoring frameworks that calculate geometric means of multiple confidence factors including OCR confidence scores, AI analysis confidence scores, and data source reliability scores. The uncertainty quantification framework may incorporate measures of agreement between multiple artificial intelligence models used in analyzing the ingredient information, wherein disagreement between models may reduce overall confidence scores while consensus may increase confidence levels. These technical implementations may address computational challenges in assessing the reliability of automated analysis results and providing users with quantified measures of recommendation certainty.

The output generation module 514 may implement weighted scoring algorithms that apply different priority levels to various types of health implications based on severity and specificity of conditions in the user health profile data 518. In some embodiments, the module 514 may treat allergen-related implications with higher priority than general nutritional concerns through algorithmic weighting systems that ensure potentially dangerous ingredient interactions receive appropriate computational emphasis. The module 514 may implement hierarchical display systems that aggregate ingredient-level indicators using weighted algorithms to determine overall product-level compatibility assessments, where critical allergens may override otherwise acceptable ingredients through rule-based logic systems.

The personalized ingredient analysis system 500 may implement real-time API integration frameworks that enable dynamic retrieval of updated regulatory status information from multiple authoritative health databases simultaneously, including FDA GRAS databases, NIH compound databases, and international regulatory databases. The system 500 may employ caching mechanisms that store frequently accessed ingredient information locally while maintaining synchronization with remote databases through scheduled updates, addressing technical challenges in balancing data currency with system performance. These technical features may solve computational problems related to maintaining accurate and current health information across distributed database systems while providing responsive user experiences.

Embodiments of the present invention may implement adaptive learning mechanisms that incorporate user feedback to refine future comparison processes through machine learning algorithms that adjust sensitivity thresholds and matching criteria based on user interactions. The system 500 may employ conflict resolution algorithms that prioritize higher severity ratings when conflicting information is retrieved from different authoritative sources, implementing automated decision-making processes for handling inconsistent data across multiple health databases. These technical implementations may address computational challenges in maintaining system accuracy and reliability while adapting to evolving user needs and changing health information landscapes.

It is to be understood that although the invention has been described above in terms of particular embodiments, the foregoing embodiments are provided as illustrative only, and do not limit or define the scope of the invention. Various other embodiments, including but not limited to the following, are also within the scope of the claims. For example, elements and components described herein may be further divided into additional components or joined together to form fewer components for performing the same functions.

Any of the functions disclosed herein may be implemented using means for performing those functions. Such means include, but are not limited to, any of the components disclosed herein, such as the computer-related components described below.

The techniques described above may be implemented, for example, in hardware, one or more computer programs tangibly stored on one or more computer-readable media, firmware, or any combination thereof. The techniques described above may be implemented in one or more computer programs executing on (or executable by) a programmable computer including any combination of any number of the following: a processor, a storage medium readable and/or writable by the processor (including, for example, volatile and non-volatile memory and/or storage elements), an input device, and an output device. Program code may be applied to input entered using the input device to perform the functions described and to generate output using the output device.

Embodiments of the present invention may utilize a wide variety of artificial intelligence models and architectures to perform ingredient analysis and health implication determination. The AI analysis module 510 may implement neural network architectures that include feedforward neural networks, convolutional neural networks (CNNs), recurrent neural networks (RNNs), and transformer-based architectures. In some cases, the AI analysis module 510 may employ deep learning models with multiple hidden layers, such as networks containing at least 10 layers, at least 50 layers, at least 100 layers, or at least 1,000 layers, enabling complex pattern recognition and feature extraction from ingredient data. The neural networks may contain at least 1 million parameters, at least 10 million parameters, at least 100 million parameters, at least 1 billion parameters, or at least 10 billion parameters to provide sufficient computational capacity for processing complex ingredient relationships and generating nuanced health implications 526.

The AI analysis module 510 may implement transformer-based models that utilize self-attention mechanisms to analyze ingredient sequences and contextual relationships within food products. These transformer architectures may include encoder-only models, decoder-only models, and encoder-decoder configurations that process ingredient information through multi-head attention layers. In some embodiments, the AI analysis module 510 may employ autoregressive language models that generate health implications 526 by predicting subsequent tokens based on ingredient input sequences. The transformer models may utilize positional encoding schemes, layer normalization techniques, and residual connections to maintain information flow through deep network architectures. The AI analysis module 510 may implement attention mechanisms that process at least 512 tokens, at least 1,024 tokens, at least 4,096 tokens, at least 8,192 tokens, or at least 32,768 tokens simultaneously, enabling comprehensive analysis of complex ingredient lists and their associated health implications.

The AI analysis module 510 may utilize large language models (LLMs) from various model families to process ingredient information and generate health implications 526. In some cases, the AI analysis module 510 may employ models from the GPT family, including GPT-3, GPT-3.5, GPT-4, GPT-4 Turbo, and future iterations of the GPT architecture. The module 510 may alternatively or additionally implement models from the Claude family, such as Claude-1, Claude-2, Claude-3 Haiku, Claude-3 Sonnet, and Claude-3 Opus, which may provide specialized capabilities for analyzing ingredient safety and health implications. The AI analysis module 510 may utilize models from the Gemini family, including Gemini Pro, Gemini Ultra, and Gemini Nano, which may offer multimodal processing capabilities for analyzing both textual ingredient information and visual packaging data. In some embodiments, the AI analysis module 510 may employ models from the LLAMA family, such as LLAMA-2, Code Llama, and specialized fine-tuned variants optimized for nutritional and health data analysis.

The AI analysis module 510 may implement specialized domain-specific models that have been fine-tuned on nutritional and medical datasets. These models may include BioBERT, ClinicalBERT, PubMedBERT, and other biomedical language models that have been pre-trained on scientific literature and medical texts. In some cases, the AI analysis module 510 may utilize models such as SciBERT, which has been trained on scientific publications, or ChemBERTa, which has been specifically designed for chemical and molecular analysis tasks relevant to ingredient processing. The module 510 may employ models from the T5 family, including T5-Small, T5-Base, T5-Large, T5-3B, and T5-11B, which utilize text-to-text transfer learning approaches for generating health implications from ingredient inputs. The AI analysis module 510 may implement BERT-based models, including BERT-Base, BERT-Large, RoBERTa, DeBERTa, and ELECTRA, which may provide bidirectional encoding capabilities for understanding ingredient contexts and relationships.

The AI analysis module 510 may utilize ensemble methods that combine multiple model architectures to improve accuracy and reliability of health implication determinations. The ensemble approaches may include model averaging techniques, weighted voting systems, stacking methods, and boosting algorithms that aggregate predictions from multiple AI models. In some embodiments, the AI analysis module 510 may implement mixture of experts (MoE) architectures that route different types of ingredient analysis tasks to specialized sub-models within a larger framework. The module 510 may employ federated learning approaches that combine knowledge from multiple distributed models while maintaining data privacy and security requirements. The AI analysis module 510 may utilize multi-task learning architectures that simultaneously perform ingredient classification, allergen detection, nutritional analysis, and health risk assessment through shared representation learning.

The AI analysis module 510 may implement reinforcement learning models that optimize health implication generation based on user feedback and outcome data. These reinforcement learning approaches may include Q-learning algorithms, policy gradient methods, actor-critic architectures, and deep reinforcement learning techniques that adapt model behavior based on user interactions with the personalized ingredient analysis system 500. In some cases, the AI analysis module 510 may employ generative adversarial networks (GANs) that include generator networks for creating health explanations and discriminator networks for validating the accuracy and relevance of generated content. The module 510 may utilize variational autoencoders (VAEs) that learn compressed representations of ingredient data and generate probabilistic health implications based on learned latent spaces.

The AI analysis module 510 may implement graph neural networks (GNNs) that model ingredient relationships and chemical interactions through graph-structured data representations. These graph-based approaches may include graph convolutional networks (GCNs), graph attention networks (GATs), and message passing neural networks (MPNNs) that analyze ingredient connectivity patterns and molecular structures. In some embodiments, the AI analysis module 510 may employ knowledge graph embedding models such as TransE, TransR, ComplEx, and RotatE that learn vector representations of ingredient entities and their relationships within structured knowledge bases. The module 510 may utilize graph transformer architectures that combine graph-based reasoning with attention mechanisms to analyze complex ingredient interaction networks and generate comprehensive health implications 526.

The AI analysis module 510 may implement computer vision models for processing visual ingredient information captured through image capture devices. These computer vision architectures may include convolutional neural networks such as ResNet, DenseNet, EfficientNet, and Vision Transformer (ViT) models that analyze packaging images and extract ingredient information. In some cases, the AI analysis module 510 may employ object detection models such as YOLO, R-CNN, and SSD architectures that identify and localize ingredient text regions within captured images. The module 510 may utilize optical character recognition models that combine convolutional layers with recurrent architectures, such as CRNN (Convolutional Recurrent Neural Network) models, to extract text from ingredient labels with high accuracy across diverse packaging formats.

The AI analysis module 510 may implement multimodal models that process both textual and visual information simultaneously to generate comprehensive health implications 526. These multimodal architectures may include CLIP (Contrastive Language-Image Pre-training), DALL-E, GPT-4V, and other vision-language models that understand relationships between ingredient text and packaging imagery. In some embodiments, the AI analysis module 510 may employ cross-modal attention mechanisms that align textual ingredient descriptions with visual packaging elements to improve analysis accuracy and completeness. The module 510 may utilize multimodal transformer architectures that process ingredient data through separate encoding pathways for different input modalities before combining representations through fusion layers.

The AI analysis module 510 may implement time series analysis models for processing temporal aspects of ingredient data and health implications. These temporal models may include Long Short-Term Memory (LSTM) networks, Gated Recurrent Units (GRUs), and Temporal Convolutional Networks (TCNs) that analyze ingredient consumption patterns and health outcome trends over time. In some cases, the AI analysis module 510 may employ attention-based sequence models that process ingredient interaction histories and generate time-sensitive health recommendations based on user consumption patterns. The module 510 may utilize forecasting models such as Prophet, ARIMA variants, and neural forecasting architectures that predict future health implications based on ingredient exposure patterns and user health profile changes.

The AI analysis module 510 may implement federated learning architectures that enable collaborative model training across distributed data sources while maintaining user privacy and data security. These federated approaches may include FedAvg (Federated Averaging), FedProx, and differential privacy techniques that aggregate model updates from multiple users without exposing individual health profile data 518. In some embodiments, the AI analysis module 510 may employ secure multi-party computation protocols and homomorphic encryption techniques that enable model inference on encrypted ingredient and health data. The module 510 may utilize blockchain-based consensus mechanisms for validating model updates and maintaining integrity of distributed learning processes across the personalized ingredient analysis system 500.

Any claims herein which affirmatively require a computer, a processor, a memory, or similar computer-related elements, are intended to require such elements, and should not be interpreted as if such elements are not present in or required by such claims. Such claims are not intended, and should not be interpreted, to cover methods and/or systems which lack the recited computer-related elements. For example, any method claim herein which recites that the claimed method is performed by a computer, a processor, a memory, and/or similar computer-related element, is intended to, and should only be interpreted to, encompass methods which are performed by the recited computer-related element(s). Such a method claim should not be interpreted, for example, to encompass a method that is performed mentally or by hand (e.g., using pencil and paper). Similarly, any product claim herein which recites that the claimed product includes a computer, a processor, a memory, and/or similar computer-related element, is intended to, and should only be interpreted to, encompass products which include the recited computer-related element(s). Such a product claim should not be interpreted, for example, to encompass a product that does not include the recited computer-related element(s).

Each computer program within the scope of the claims below may be implemented in any programming language, such as assembly language, machine language, a high-level procedural programming language, or an object-oriented programming language. The programming language may, for example, be a compiled or interpreted programming language.

Each such computer program may be implemented in a computer program product tangibly embodied in a machine-readable storage device for execution by a computer processor. Method steps of the invention may be performed by one or more computer processors executing a program tangibly embodied on a computer-readable medium to perform functions of the invention by operating on input and generating output. Suitable processors include, by way of example, both general and special purpose microprocessors. Generally, the processor receives (reads) instructions and data from a memory (such as a read-only memory and/or a random access memory) and writes (stores) instructions and data to the memory. Storage devices suitable for tangibly embodying computer program instructions and data include, for example, all forms of non-volatile memory, such as semiconductor memory devices, including EPROM, EEPROM, and flash memory devices; magnetic disks such as internal hard disks and removable disks; magneto-optical disks; and CD-ROMs. Any of the foregoing may be supplemented by, or incorporated in, specially-designed ASICs (application-specific integrated circuits) or FPGAs (Field-Programmable Gate Arrays). A computer can generally also receive (read) programs and data from, and write (store) programs and data to, a non-transitory computer-readable storage medium such as an internal disk (not shown) or a removable disk. These elements will also be found in a conventional desktop or workstation computer as well as other computers suitable for executing computer programs implementing the methods described herein, which may be used in conjunction with any digital print engine or marking engine, display monitor, or other raster output device capable of producing color or gray scale pixels on paper, film, display screen, or other output medium.

Any data disclosed herein may be implemented, for example, in one or more data structures tangibly stored on a non-transitory computer-readable medium. Embodiments of the invention may store such data in such data structure(s) and read such data from such data structure(s).

Any step or act disclosed herein as being performed, or capable of being performed, by a computer or other machine, may be performed automatically by a computer or other machine, whether or not explicitly disclosed as such herein. A step or act that is performed automatically is performed solely by a computer or other machine, without human intervention. A step or act that is performed automatically may, for example, operate solely on inputs received from a computer or other machine, and not from a human. A step or act that is performed automatically may, for example, be initiated by a signal received from a computer or other machine, and not from a human. A step or act that is performed automatically may, for example, provide output to a computer or other machine, and not to a human.

The terms “A or B,” “at least one of A or/and B,” “at least one of A and B,” “at least one of A or B,” or “one or more of A or/and B” used in the various embodiments of the present disclosure include any and all combinations of words enumerated with it. For example, “A or B,” “at least one of A and B” or “at least one of A or B” may mean: (1) including at least one A, (2) including at least one B, (3) including either A or B, or (4) including both at least one A and at least one B.

Although terms such as “optimize” and “optimal” are used herein, in practice, embodiments of the present invention may include methods which produce outputs that are not optimal, or which are not known to be optimal, but which nevertheless are useful. For example, embodiments of the present invention may produce an output which approximates an optimal solution, within some degree of error. As a result, terms herein such as “optimize” and “optimal” should be understood to refer not only to processes which produce optimal outputs, but also processes which produce outputs that approximate an optimal solution, within some degree of error.

Unless expressly and specifically stated otherwise in this specification, the omission from this specification of any subject matter, terminology, embodiments, examples, features, elements, steps, or other content that was disclosed in any application to which this application claims priority (including, but not limited to, any provisional application) is not intended to disclaim, surrender, or narrow the scope of any claim term herein. Such omissions are made solely for purposes of brevity, clarity, organization, or drafting preference and shall not be construed as evidencing any intent by the applicant to limit, restrict, or abandon any aspect of the claimed invention or to exclude any interpretation that would otherwise be available based on the incorporated subject matter. The applicant specifically reserves the right to claim the full scope of any invention disclosed in any application incorporated herein by reference or otherwise whose priority or benefit is claimed, whether or not such invention is explicitly redescribed in this specification. Any construction of claim terms should consider the full scope of disclosure available in this specification together with all incorporated applications, and no negative inference should be drawn from any omission of previously disclosed subject matter unless such limitation is expressly and unambiguously set forth in this specification.

1. A computer-implemented method for providing personalized ingredient analysis, comprising:
(A) receiving user health profile data including at least one of dietary restrictions, allergies, or health conditions;
(B) capturing ingredient data from a food product using at least one of:
an image capture device associated with a computing device;
a barcode scanner associated with the computing device; or
an RFID tag reader associated with the computing device;
(C) deriving ingredient information from the captured ingredient data, the ingredient information representing a plurality of identified ingredients, wherein deriving the ingredient information comprises at least one of:
performing optical character recognition (OCR) processing on captured image data to extract text,
deriving the ingredient information based on a QR code in the captured image data;
deriving the ingredient information based on a scanned barcode; or
deriving the ingredient information based on a read RFID tag identifier;
(D) analyzing the ingredient information using an artificial intelligence to determine system health implications, wherein the artificial intelligence system comprises at least one of a natural language processing model or a machine learning model trained on nutritional and health data, wherein the artificial intelligence system utilizes ontological reasoning to infer implicit relationships between ingredients and health conditions, including identifying that an ingredient belongs to a broader class of compounds associated with allergen categories;
(E) comparing the health implications against the user health profile data using a three-tier hierarchy of priority rules to produce comparison output, wherein the three-tier hierarchy comprises allergen-related implications having highest priority, additive-related implications having medium priority, and preference-related implications having lowest priority, wherein comparing using the three-tier hierarchy comprises implementing weighted scoring algorithms that assign different priority levels to the health implications based on the three-tier hierarchy, and wherein critical allergens override otherwise acceptable ingredients;
(F) calculating a confidence score for the comparison output;
(G) when the confidence score exceeds a predetermined threshold:
generating an output based on the comparison output, wherein the output includes a visual Go/No-Go indicator representing compatibility between the user health profile data and at least one of: (1) the food product; or (2) at least one of the plurality of identified ingredients; and
providing the output to a user; and
(H) when the confidence score does not exceed the predetermined threshold, not generating the output based on the comparison output.

2. The method of claim 1:
wherein capturing the ingredient data from the food product comprises using the image capture device to capture an image from the food product; and
wherein deriving the ingredient information from the captured image data comprises:
performing OCR processing on the captured image to extract text from the captured image; and
deriving the ingredient information from the extracted text.

3. The method of claim 2, wherein deriving the ingredient information from the extracted text comprises:
tokenizing the extracted text into a plurality of tokens;
identifying ingredient names using a predefined ingredient database; and
associating descriptors with their corresponding ingredients.

4. The method of claim 2, wherein parsing the extracted text comprises:
tokenizing the extracted text into a plurality of tokens;
identifying ingredient names using natural language processing; and
associating descriptors with their corresponding ingredients.

5. The method of claim 1:
wherein capturing the ingredient data from the food product comprises using the barcode scanner to scan a barcode from the food product; and
wherein deriving the ingredient information from the captured ingredient data comprises deriving the ingredient information based on the scanned barcode.

6. The method of claim 1:
wherein capturing the ingredient data from the food product comprises using the RFID tag reader to read an RFID tag identifier from the food product; and
wherein deriving the ingredient information from the captured ingredient data comprises deriving the ingredient information based on the RFID tag identifier.

7. The method of claim 1, wherein analyzing the ingredient information using the artificial intelligence system comprises:
(D) (1) inputting the ingredient information into the natural language processing model; and
(D) (2) generating the health implications using the natural language processing model.

8. The method of claim 1, wherein analyzing the ingredient information using the artificial intelligence system comprises:
(D) (1) inputting the ingredient information into the machine learning model trained on nutritional and health data; and
(D) (2) generating the health implications based on outputs from the machine learning model.

9. The method of claim 8, wherein the machine learning model comprises a large language model fine-tuned on a corpus of nutritional and medical literature.

10. The method of claim 8, wherein generating the health implications comprises:
(D) (2) (a) determining potential allergen risks associated with each of the identified ingredients represented by the ingredient information;
(D) (2) (b) identifying potential interactions between the plurality of identified ingredients and medications listed in the user health profile data; and
(D) (2) (c) assessing nutritional impacts of the plurality of ingredients based on dietary restrictions or health conditions in the user health profile data.

11. The method of claim 1, wherein analyzing the ingredient information using the artificial intelligence system comprises:
generating plain language explanations of the determined health implications for each of the plurality of identified ingredients.

12. The method of claim 1, wherein analyzing the ingredient information using the artificial intelligence system comprises:
assigning a risk score to each of the plurality of identified ingredients based on the determined health implications and the user health profile data.

13. The method of claim 12, further comprising:
calculating an overall risk score for the food product based on the risk scores of the plurality of identified ingredients.

14. The method of claim 1, wherein analyzing the ingredient information using the artificial intelligence system comprises:
categorizing each of the plurality of identified ingredients into one of multiple risk levels based on the determined health implications and the user health profile data.

15. The method of claim 1, wherein analyzing the ingredient information using the artificial intelligence system comprises:
identifying potential cumulative effects or interactions between multiple ingredients in the plurality of identified ingredients.

16. The method of claim 1, wherein comparing the health implications against the user health profile data comprises:
(E) (1) matching each identified ingredient against a list of allergens in the user health profile data; and
(E) (2) flagging any ingredients that match allergens in the user health profile data.

17. The method of claim 1, wherein comparing the health implications against the user health profile data comprises:
(E) (1) analyzing potential interactions between the identified ingredients and medications listed in the user health profile data; and
(E) (2) generating alerts for any identified potential interactions.

18. The method of claim 1, wherein comparing the health implications against the user health profile data comprises:
(E) (1) calculating a compatibility score for each of the plurality of identified ingredients based on the user health profile data, thereby calculating a plurality of compatibility scores; and
(E) (2) determining an overall compatibility score for the food product based on the plurality of compatibility scores.

19. The method of claim 1, wherein comparing the determined health implications against the user health profile data comprises:
(E) (1) assigning sensitivity levels to different identified ingredients based on the user health profile data; and
(E) (2) adjusting risk assessments for the plurality of identified ingredients based on the assigned sensitivity levels.

20. A system comprising at least one non-transitory computer-readable medium having computer program instructions stored thereon, the computer-program instructions being executable by at least one computer processor to perform a method for providing personalized ingredient analysis, the method comprising:
(A) receiving user health profile data including at least one of dietary restrictions, allergies, or health conditions;
(B) capturing ingredient data from a food product using at least one of:
an image capture device associated with a computing device;
a barcode scanner associated with the computing device; or
an RFID tag reader associated with the computing device;
(C) deriving ingredient information from the captured ingredient data, the ingredient information representing a plurality of identified ingredients, wherein deriving the ingredient information comprises at least one of:
performing optical character recognition (OCR) processing on captured image data to extract text,
deriving the ingredient information based on a QR code in the captured image data;
deriving the ingredient information based on a scanned barcode; or
deriving the ingredient information based on a read RFID tag identifier;
(D) analyzing the ingredient information using an artificial intelligence system to determine health implications, wherein the artificial intelligence system comprises at least one of a natural language processing model or a machine learning model trained on nutritional and health data, wherein the artificial intelligence system utilizes ontological reasoning to infer implicit relationships between ingredients and health conditions, including identifying that an ingredient belongs to a broader class of compounds associated with allergen categories;
(E) comparing the health implications against the user health profile data using a three-tier hierarchy of priority rules to produce comparison output, wherein the three-tier hierarchy comprises allergen-related implications having highest priority, additive-related implications having medium priority, and preference-related implications having lowest priority, wherein comparing using the three-tier hierarchy comprises implementing weighted scoring algorithms that assign different priority levels to the health implications based on the three-tier hierarchy, and wherein critical allergens override otherwise acceptable ingredients;
(F) calculating a confidence score for the comparison output;
(G) when the confidence score exceeds a predetermined threshold:
generating an output based on the comparison output, wherein the output includes a visual Go/No-Go indicator representing compatibility between the user health profile data and at least one of: (1) the food product; or (2) at least one of the plurality of identified ingredients; and
providing the output to a user; and
(H) when the confidence score does not exceed the predetermined threshold, not generating the output based on the comparison output.

21. The method of claim 1, further comprising displaying a message indicating insufficient confidence when the confidence score does not exceed the predetermined threshold.

22. The system of claim 20, further comprising displaying a message indicating insufficient confidence when the confidence score does not exceed the predetermined threshold.
